(19) United States
(12) Patent Application Publication
(10) Pub. No.: US 2009/0012024 A1
(43) Pub. Date: Jan. 8, 2009

(54) Stem Cell Markers

(75) Inventors: Anne Collins, York (GB); Norman Maitland, York (GB); Steven Bryce, York (GB)

Correspondence Address: CROWELL & MORING LLP;INTELLECTUAL PROPERTY GROUP, P.O. BOX 14300, WASHINGTON, DC, 20044-4300, US

(73) Assignee: Procure Therapeutics Limited, York (GB)

(21) Appl. No.: 11/996,839

(22) PCT Filed: Jul. 19, 2006
(86) PCT No.: PCT/GB2006/002658

§ 371 Date: Sep. 11, 2008

(30) Foreign Application Priority Data
Jul. 26, 2005 (GB) 0515305.1
Jul. 26, 2005 (GB) 0515307.7
Jul. 26, 2005 (GB) 0515308.5
Jul. 26, 2005 (GB) 0515309.3

(51) Int. Cl. A61K 31/711; C07K 16/18; C12N 15/85; C12N 5/12; C12P 21/02; C12Q 1/68; A61P 35/00

(52) U.S. Cl. 514/44; 530/387.9; 530/387.3; 536/23.5; 435/328; 435/69.6; 435/320.1

(57) ABSTRACT

We disclose gene markers of stem cells, typically prostate stem cells, and in particular cancer stem cells, for example prostate cancer stem cells; therapeutic agents and diagnostic assays based on said stem cell genes; and including screening assays to identify therapeutic agents.

The invention relates to gene markers of stem cells, typically prostate stem cells, and in particular cancer stem cells, for example prostate cancer stem cells; therapeutic agents and diagnostic assays based on said stem cell genes and expression products; and including screening assays to identify therapeutic agents.

### BACKGROUND

A problem underlying the effective treatment of cancerous conditions is the identification of a population of cells in a tumour that have the ability of sustaining the growth of a tumour. The evidence suggests that tumours are clonal and are therefore derived from a single cell. However, there are few studies that identify and characterise those cells types that are responsible for maintaining tumour cell growth. Some have searched for these so called “cancer stem cells”.

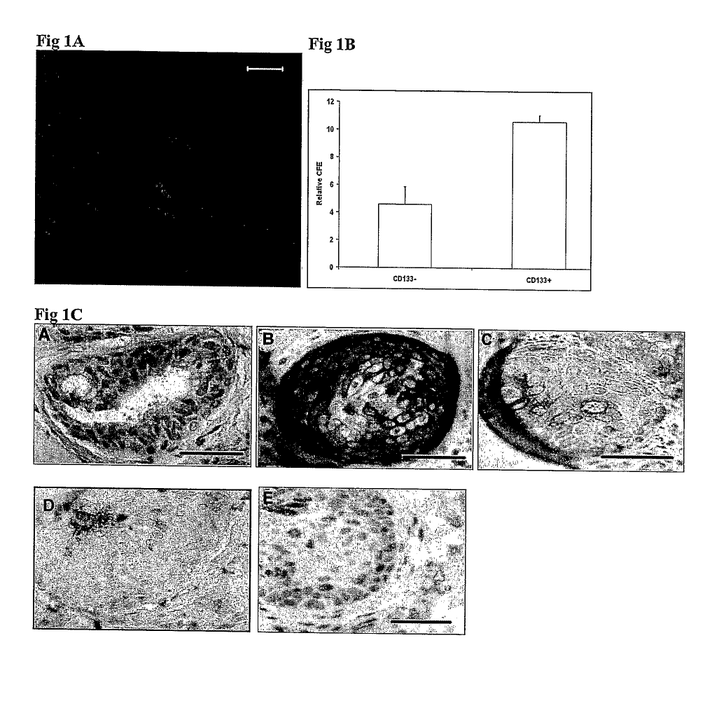

We have identified CD133, which is expressed by primitive haematopoietic stem cells and developing epithelia as a further stem cell marker for prostate epithelia. CD133 cells are restricted to the α₂β₁^{hi} population (the receptor for type I collagen) and are located in the basal layer, often at the base of a budding region or branching point (FIG. 1A). α₂β₁^{hi}/CD133⁺ cells exhibit two important attributes of epithelial stem cells: they possess a high in vitro proliferative potential (FIG. 1B) and can reconstitute prostatic-like acini in immunocompromised male nude mice FIG. 1C).

In our co-pending application (WO 2005/089043) we describe the isolation of prostate stem cells which have been directly isolated from lymph node and prostate glands from a series of patient samples. These stem cells express markers that characterise the cells with stem cell properties. The following markers are typically expressed as prostate stem cell markers; human epithelial antigen (HEA), CD44, α₂β₁^{hi} and CD133. Morphologically the cells range from fibroblastoid (expressing high levels of vimentin which is typical of transformed cells) or epithelial, and are capable of producing progenitors associated with prostate epithelial differentiation. Invasion assays, using Matrigel-coated filters have determined that these cells have 2-3 fold greater capacity to invade through Matrigel than PC3M (a highly metastatic sub-line of PC3 cells).

### DESCRIPTION OF THE DISCLOSURE

We have conducted array analysis of CD133 positive prostate stem cells and compared expression of genes between CD133 positive stem cells and CD133 positive cancer stem cells.

### Growth Factors/Receptors

The array analysis has identified a number of genes that encode proteins that are either growth factors/receptors or proteins involved in signal transduction pathways that result in stimulation of cell growth and/or cell proliferation. A group of growth factors, referred to as cytokines, are involved in a number of diverse cellular functions. These include, by example and not by way of limitation, modulation of the immune system, regulation of energy metabolism and control of growth and development. Cytokines mediate their effects via receptors expressed at the cell surface on target cells. Cytokine receptors can be divided into three separate sub groups. Type 1 (e.g. growth hormone (GH) family) receptors are characterised by four conserved cysteine residues in the amino terminal part of their extracellular domain and the presence of a conserved Trp-Ser-Xaa-Trp-Ser motif in the C-terminal part. The repeated Cys motif is also present in Type 2 (interferon family) and Type III (tumour necrosis factor family).

A further group of growth factors are involved in angiogenesis. Angiogenesis is the development of new blood vessels from an existing vascular bed and is a complex multistep process that involves the degradation of components of the extracellular matrix and then the migration, cell-division and differentiation of endothelial cells to form tubules and eventually new vessels. Angiogenesis is involved in pathological conditions such as tumour cell growth. Genes involved in angiogenesis include, by example and not by way of limitation; vascular endothelial growth factor (VEGF, VEGF B, VEGF C, VEGF D); transforming growth factor (TGFb); acidic and basic fibroblast growth factor (AFGF and bFGF); and platelet derived growth factor (FDGF).

Many receptors for growth factors and the like are membrane bound via glycosylphosphatidylinositol (GPI). GPI-anchors are post-translational modifications to proteins that add glycosylphosphatidylinositol which enable these proteins to anchor to the extracellular side of cell membranes. Typically, extracellular proteins which have a GPI anchor do not have transmembrane or cytoplasmic domains. GPI anchor proteins occur in all eulcaryotes and form a diverse variety of proteins that includes, for example, membrane associated enzymes and adhesion molecules.

Genes that are involved in signal transduction are often associated with cell membrane localised receptors which upon activation results in a signal transduction cascade leading to activation of transcription and cell proliferation. For example, the PTEN gene product is a multiple-specificity phosphatase that can antagonise phosphoinositide lipid kinases (hereinafter PI3K) by degrading phosphoinositide (PI) (3,4,5) P3 back to PI(4,5)P2 in addition to its ability to dephosphorylate a range of protein targets such as focal adhesion kinase OAK). Phosphoinositides have been implicated in a variety of cellular processes as diverse as vacuolar protein sorting, cytoskeletal remodelling and mediating intracellular signalling events through which growth factors, hormones and neurotransmitters exert their physiological effects on cellular activity, proliferation and differentiation.

### Extracellular Matrix Associated

The array analysis has identified a number of genes that encode proteins that are extracellular matrix proteins. The Extracellular Matrix (ECM) is a complex mixture of non-living material which surrounds cells in multicellular organisms. In vertebrates the ECM comprises a mixture of protein and carbohydrate (and minerals in the example of bone). The protein component comprises proteins and glycoproteins proteins that are modified by the addition of sugar moieties) such as collagens (collagens I-XII); laminins which are found in the basal lamina a structure to which epithelial cells associate; fibronectin which functions to bind cells to the ECM; and elastins which provide skin with the flexibility. Proteoglycans are also found in the ECM and these glycoproteins comprise more carbohydrate than protein. Several sugars are added to proteoglycans the most abundant of which is acetylglucosamine. Many proteoglycans are sulphated, for example chondroitin sulphate, heparan sulphate, keratin sulphate and hyaluronic acid.

Most vertebrate cells cannot survive unless associated with the ECM and the loss of anchorage to the ECM is often associated with cell transformation in cancer. Cells attach to the ECM via anchoring molecules expressed by the cell called intergrins which bind the ECM via collagen, laminins and fibronectin. Integrins are cell membrane proteins that bind the extracellular matrix via their extracellular domain which projects from the cell surface. The integrin intracellular domain contacts the actin filaments of the cytoskeleton. In cancer metastasis the primary cancer cell becomes motile and is able to metastase (transfer) to other tissues to form secondary cancers. It is the secondary cancer that eventually kills the subject. In order to metastase the cancer cell has to penetrate the basement membrane of the tissue to gain access to the blood and lymph systems which transports the cancer cell around the body. The degradation of the ECM around a tumour is mainly catalysed by metalloproteases (MMP) which are secreted by stromal cells that surround the tumour. There is increasing evidence that cancer cells stimulate MMP production by fibroblasts by the paracrine secretion of hormones.

MMP's are an expanding group of proteases that can be classified into 3 groups. Group 1 includes collagenases that degrade connective tissue collagen, for example MMP-1, MMP-8 and MMP-13; group 2 includes gelatinases that degrade basement membrane collagens and include MMP-2 and MMP-9; a third group includes the stromelysins that degrade ECM proteoglycans, laminin and fibronectin and include MMP-10 and M-11. The activity of MMP's can be enhanced by pro-inflammatory cytokines such as IL-1 and TNFα.

### Transcription Factors/Transcription Related

The array analysis has identified a number of genes that encode proteins that are transcription factor proteins or proteins that are related to transcription.

Transcription factors are proteins that bind to DNA enhancer or promoter elements. Often these are near to the start of transcription of a gene. Transcription factors either inhibit or facilitate RNA polymerase transcription initiation and also the maintenance of an active transcription complex. Transcription factors contain two basic functional domains. A transactivation domain which is a region of the protein which interacts with other parts of the transcription machinery, for example the RNA polymerase or other transcription factors; and a DNA binding domain which comprises amino acids which recognise specific bases within the promoter region of the gene. In some examples enhancer elements can be positioned at a distance from the start of transcription or even within introns, Lewin B, 1994. Genes V, Oxford University Press, Oxford. Frequently DNA binding domains interact with nucleotide sequences or motifs which are sites to which the transcription factor binds to enhance or repress transcription.

An important family of the transcription factors comprises the homeodomain proteins. This family of transcription factors is characterised by the so-called homeodomain region which consists of 16 amino acids arranged in a helix-turn-helix conformation. Some transcription factors have both a homeodomain and a second DNA-binding region. In some examples, the region that comprises the homeodomain and the second DNA-binding region is called the POU domain.

A further example of a family of transcription factors which contain a conserved binding domain is the helix-loop-helix domain. The muscle specific transcription factor MyoD contains this motif, as do several D. melanogaster proteins that determine the cell fate in the D. melanogaster peripheral nervous system.

A related family of transcription factors are referred to as the basic leucine zipper transcription factor family or bZip. The bZip proteins are dimers, each of whose subunits contain a basic DNA-binding domain at the carboxyl end followed closely by a helix containing several leucine residues. Examples of bZip family members are C/EBP, Ap1, and the yeast transcription factor GCN4

A large group of transcription factors are the nuclear hormone receptors. It is known that steroid hormones increase the transcription of specific groups of genes. Once the hormone has entered the cell, it binds to its specific receptor protein, converting that receptor into a conformation that is able to enter the nucleus and bind specific DNA sequence motifs. The family of steroid hormone receptors includes proteins that recognise oestrogen, progesterone, testosterone and cortisone as well as non-steroid lipids such as retinoic acid, thyroxine and vitamin D.

It is known that the way in which DNA is packaged as chromatin can influence the expression of genes. There are several levels of structural packaging of DNA leading from a double stranded helix to a mitotic chromosome, after which the DNA is some 50,000 times shorter than its extended length. Double-stranded helical DNA is wound around the structural unit of a nucleosome, comprising an octamer core composed of 4 types of histones: two each of the H2A, H2B, H3, and H4 proteins. Approximately 166 base pairs are bound to the nucleosome through electrostatic forces between the negatively charged phosphate groups in the DNA backbone and positively charged amino acids (e.g., lysine and arginine) in the histone proteins.

Nucleosomes are organised into the next structural level of the chromatin fibre, also referred to as a solenoid. Chromatin structure is not static and the regulated alteration in structure is termed ‘chromatin remodelling’. This process has been defined as any event that alters the nuclease sensitivity of a region of chromatin, and can occur independently or in concert with processes such as transcription.

Reversible acetylation of evolutionary conserved lysine residues in core histone proteins plays a critical role in transcriptional regulation, cell cycle progression, and developmental events. The steady state of histone acetylation is controlled by the enzymatic activities of multiple histone acetyltransferases (HATs) and histone deacetylases (HDACs). Histone hyperacetylation is associated with transcriptional activity while histone hypoacetylation correlates with transcriptional quiescence and so histone deacetylases can be considered as enzymatic transcriptional repressors.

In general, histone deacetylases do not target genes directly through specific DNA-binding sites. Rather, deacetylases are localized to genes targeted for repression as part of a protein complex. Other proteins that are part of this complex, termed co-repressors, are responsible for targeting the genes to be repressed. In humans, four highly homologous class I HDAC enzymes (HDAC1, HDAC2, HDAC3, and HDAC8) have been identified to date, with HDAC1, HDAC2 and HDAC3 being ubiquitously expressed in many different cell types (Yang et al., 1997 and 2002). HDAC1 and HDAC2 are the human orthologues of the yeast transcriptional regulator RPD3. Analysis of the predicted amino acid sequence of HDAC3 revealed an open reading frame of 428 amino acids with a predicted molecular mass of 49 kDa.

### DNA Replication and Repair

The array analysis has identified a number of genes that encode proteins that are involved in DNA replication or repair of DNA. DNA damage can occur through a number of agents. For example, certain wavelengths of radiation, (e.g. gamma rays or X-rays), ultraviolet rays especially UV-C rays that are absorbed strongly by DNA; highly reactive oxygen radicals produced during respiration and other metabolic processes; and chemical mutagens found in the environment which may be man made or naturally occurring.

DNA can be damaged in different ways. For example, the four bases that form DNA can be covalently modified at various positions; deamination of an amino group is a common modification resulting in a mutation of cytosine to uracil. Other modifications include mismatches, for example the conversion of thymidine to uracil, single strand breaks in the phosphate backbone of the DNA molecule and covalent crosslinks between bases which may be intra-strand or inter-strand. Several chemotherapeutic agents used in the treatment of cancer act as crosslinking agents.

The Human Genome Project which has deduced the complete DNA sequence of human DNA has identified around 130 genes thought to be involved in DNA repair and replication. Damaged or inappropriate incorporation of bases can be corrected via several mechanisms. These include direct chemical reversal or excision repair. Excision repair results in removal of the damaged base and replacement with the correct base. There are three mechanisms of excision repair utilised by cells to repair DNA damage.

Base excision repair involves the removal of the damaged base by a DNA glycosylase; removal of its deoxyribose phosphate to produce gapped DNA; replacement of the correct nucleotide by a DNA polymerase β and ligation of the strand break by a DNA ligase.

Nucleotide excision repair involves recognition of the error by one or more protein factors; separating the DNA strands to produce a “bubble” by an enzyme called transcription factor IIH; scission at the 5′ and 3′ sides of the damaged area; replacement synthesis of the damaged area by DNA polymerases ε and σ; and ligation of the strand break by a DNA ligase.

Mismatch repair corrects mismatches in normal bases. The correction of mismatches utilises enzymes involved in base excision repair and proteins that recognise the mismatch for example proteins encoded by MSH2 and scission around the mismatch by MLH 1 and other proteins. A mutation in either of these genes has been associated with an inherited form of colon cancer. The repair of the mismatch is completed by the DNA polymerases ε and σ.

In addition the repair of single and double strand breaks in DNA also involves a number of proteins. The repair of single strand breaks utilises many of the proteins involved in base excision repair. A double strand break is repaired either by direct ligation of the free ends of the break or by homologous recombination. Errors in direct ligation are associated with certain cancers, for example Burkitt's lymphoma and B-cell leukaemia. The BRCA-1 and BRCA-2 genes function in homologous recombination and mutations in these genes are associated with breast and ovarian cancer.

We have conducted gene array analysis to identify genes that are characteristic of cancer stem cells which show an up regulation when compared to control stem cell samples from normal or benign stem cell populations. We herein disclose these genes and their use in the identification of therapeutic agents useful in the treatment of cancer, in particular prostate cancer, and in the development of diagnostic assays for the detection of the early on set of tumour cell growth. The present disclosure relates to the identification of cancer stem cell specific genes.

### STATEMENTS OF INVENTION

According to a first aspect of the invention there is provided agent that modulates the activity of a cancer stem cell specific nucleic acid molecule, or a polypeptide encoded by a cancer stem nucleic acid molecule, wherein said cancer cell specific nucleic acid molecule is selected from the group consisting of:
i) a nucleic acid molecule consisting of a nucleic acid sequence as represented in SEQ ID NO: 1-452;
ii) a nucleic acid molecule consisting of a nucleic acid sequence as represented in Table 1 by Genbank accession number;
iii) a nucleic acid molecule that hybridises under stringent hybridisation conditions to the nucleic acid molecule in (i) or (ii) above and which encodes a polypeptide wherein said polypeptide is stem cell specific, characterised in that said agent is for use as a pharmaceutical.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Allows Sequences that Share at Least 80% Identity to Hybridize)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Allows Sequences that Share at Least 50% Identity to Hybridize)

Hybridization: 6×SSC at RT to 55° C. for 16-20 hours

Wash at least twice: 2×-3×SSC at RT to 55° C. for 20-30 minutes each.

In a preferred embodiment of the invention said agent is an antagonist. Alternatively, said agent is an agonist.

According to a further aspect of the invention there is provided an agent that modulates the activity of a polypeptide comprising an amino acid sequence encoded by a nucleic acid molecule selected from the group consisting of:
i) a nucleic acid molecule as represented in SEQ ID NO: 1-452,
ii) a nucleic acid molecule that encodes a variant polypeptide wherein said variant polypeptide is modified by addition, deletion or substitution of at least one amino acid residue of the amino acid sequence encoded by a nucleic acid sequence selected from the group consisting of SEQ ID NO: 1-452 wherein said polypeptide is stem cell specific;
iii) a nucleic acid molecule that encodes a polypeptide consisting of an amino acid sequence as represented in Table 1 by Genbank accession number; characterised in that said agent is for use as a pharmaceutical.

A variant polypeptide may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan.

In addition, the invention features polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequence illustrated herein.

In a preferred embodiment of the invention said agent is a polypeptide. Preferably said polypeptide is an antibody or an active binding part of an antibody.

In a preferred embodiment of the invention said antibody is a monoclonal antibody or active binding part thereof.

In a preferred embodiment of the invention said antibody is a chimeric antibody or a humanised antibody produced by recombinant methods to contain the variable region of said antibody with an invariant or constant region of a human antibody.

Chimeric antibodies are recombinant antibodies in which all of the V-regions of a mouse or rat antibody are combined with human antibody C-regions. Humanised antibodies are recombinant hybrid antibodies which fuse the complimentarity determining regions from a rodent antibody V-region with the framework regions from the human antibody V-regions. The complimentarily determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen. Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not illicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In a preferred embodiment of the invention said agent is an antibody fragment.

Various fragments of immunoglobulin or antibodies are known in the art, i.e., Fab, Fab₂, F(ab′)₂, Fv, Fc, Fd, scFvs, etc. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact immunoglobulin molecule. A Fab₂ fragment comprises two joined Fab fragments. When these two fragments are joined by the immunoglobulin hinge region, a F(ab′)₂ fragment results. An Fv fragment is multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain variable region, or a fragment thereof containing the three CDRs of the heavy chain variable region, without an associated light chain moiety; and multi specific antibodies formed from antibody fragments, this has for example been described in U.S. Pat. No. 6,248,516. Fv fragments or single region (domain) fragments are typically generated by expression in host cell lines of the relevant identified regions. These and other immunoglobulin or antibody fragments are within the scope of the invention and are described in standard immunology textbooks such as Paul, Fundamental Immunology or Janeway et al. Immunobiology (cited above). Molecular biology now allows direct synthesis (via expression in cells or chemically) of these fragments, as well as synthesis of combinations thereof.

It is possible to create single variable regions, so called single chain antibody variable region fragments (scFv's). If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scfv's. Alternatively said fragments are “domain antibody fragments”. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety.

In a preferred method of the invention said antibody fragment is a single chain antibody variable region fragment.

A fragment of an antibody or immunoglobulin can also have bispecific function binding two different epitopes of two different antigens.

Preferably said chimeric/humanised monoclonal antibody to said polypeptide is produced as a fusion polypeptide in an expression vector suitably adapted for transfection or transformation of prolcaryotic or eukaryotic cells.

In a further preferred embodiment of the invention said antibodies are opsonic antibodies.

Phagocytosis is mediated by macrophages and polymorphic leukocytes and involves the ingestion and digestion of micro-organisms, damaged or dead cells, cell debris, insoluble particles and activated clotting factors. Opsonins are agents which facilitate the phagocytosis of the above foreign bodies. Opsonic antibodies are therefore antibodies which provide the same function. Examples of opsonins are the Fe portion of an antibody or compliment C3.

Preferably, said antibody is provided with a marker including a conventional label or tag, for example a radioactive and/or fluorescent and/or epitope label or tag.

In an alternative preferred embodiment of the invention said antibody, or antibody fragment had associated therewith or crosslinked thereto a therapeutic agent. Preferably said therapeutic agent is a chemotherapeutic agent.

Preferably said agent is selected from the group consisting of: cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; and camptothecin.

In an alternative preferred embodiment of the invention said agent is a nucleic acid molecule. For example, an antisense nucleic acid; an aptamer; or a small interfering RNA.

In a preferred embodiment of the invention said nucleic acid molecule is a small interfering RNA.

A technique to specifically ablate gene function is through the introduction of double stranded RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically derived from exons of the gene which is to be ablated.

The mechanism of RNA interference is being elucidated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA.

According to a further aspect of the invention there is provided a pharmaceutical composition comprising an agent according to the invention.

According to a further aspect of the invention there is provided a composition comprising a nucleic acid molecule selected from the group consisting of:
i) a nucleic acid molecule consisting of a nucleic acid sequence as represented in SEQ ID NO: 1-452;
ii) a nucleic acid molecule consisting of a nucleic acid sequence as represented in Table 1 by Genbank accession number;
iii) a nucleic acid molecule that hybridises under stringent hybridisation conditions to the nucleic acid molecule in (i) or (ii) above and which encodes a polypeptide wherein said polypeptide is stem cell specific.

According to a further aspect of the invention there is provided a composition comprising a polypeptide comprising an amino acid sequence selected from the group consisting of:
i) a polypeptide comprising an amino acid sequence as represented in Table 1 by Genbank accession number, or a variant polypeptide wherein said variant is modified by addition, deletion or substitution of at least one amino acid residue of the amino acid sequence presented in Table 1 by Genbank accession number;
ii) a polypeptide comprising an amino acid sequence encoded by a nucleic acid molecule comprising a nucleic acid sequence as represented in SEQ ID NO 1-452;
iii) a polypeptide comprising an amino acid sequence encoded by a nucleic acid molecule that hybridises under stringent hybridisation conditions to the nucleic acid molecule in (ii) above and which encodes a polypeptide wherein said polypeptide is stem cell specific wherein said composition is for use as a vaccine.

In a preferred embodiment of the invention said composition includes an adjuvant and/or a carrier.

An adjuvant is a substance or procedure that augments specific immune responses to antigens by modulating the activity of immune cells. Examples of adjuvants include, by example only, Freunds adjuvant, muramyl dipeptides, liposomes. A carrier is an immunogenic molecule which, when bound to a second molecule, augments immune responses to the latter. Some antigens are not intrinsically immunogenic yet may be capable of generating antibody responses when associated with a foreign protein molecule such as keyhole-limpet haemocyanin or tetanus toxoid. Such antigens contain B-cell epitopes but no T cell epitopes. The protein moiety of such a conjugate (the “carrier” protein) provides T-cell epitopes which stimulate helper T-cells that in turn stimulate antigen-specific B-cells to differentiate into plasma cells and produce antibody against the antigen. Helper T-cells can also stimulate other immune cells such as cytotoxic T-cells, and a carrier can fulfill an analogous role in generating cell-mediated immunity as well as antibodies.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents (for example, cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; and camptothecin.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing antibody aerosols without resort to undue experimentation.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of antibody or nucleic acid for producing the desired response in a unit of weight or volume suitable for administration to a patient. The response can, for example, be measured by determining the signal transduction enhanced or inhibited by the composition via a reporter system, by measuring downstream effects such as gene expression, or by measuring the physiological effects of the composition. Likewise, the effects of antisense/siRNA molecules can be readily determined by measuring expression of the individual genes in cells to which an antisense/siRNA composition is added. Other assays will be known to one of ordinary skill in the art and can be employed for measuring the level of the response.

The doses of antibody or nucleic acid administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of antibody are formulated and administered in doses between 1 ng and 1 mg, and preferably between 10 ng and 100 μg, according to any standard procedure in the art. Where nucleic acids or variants thereof are employed, doses of between 1 ng and 0.1 mg generally will be formulated and administered according to standard procedures. Other protocols for the administration of the compositions will be known to one of ordinary skill in the art, in which the dose amount, schedule of injections, sites of injections, mode of administration (e.g., intra-bone) and the like vary from the foregoing. Administration of the compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Pharmaceutical compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation of antibody or nucleic acids, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a vector which is adapted for the expression of the humanised or chimeric antibodies according to the invention.

According to an aspect of the invention there is provided a cell which has been transformed or transfected with the vector encoding the humanised or chimeric antibody according to the invention

According to a further aspect of the invention there is provided a method for the production of the humanised or chimeric antibody according to the invention comprising:
(i) providing a cell transformed or transfected with a vector which comprises a nucleic acid molecule encoding the humanised or chimeric antibody according to the invention;
(ii) growing said cell in conditions conducive to the manufacture of said antibody; and
(iii) purifying said antibody from said cell, or its growth environment.

In a yet further aspect of the invention there is provided a hybridoma cell line which produces a monoclonal antibody as hereinbefore described.

In a further aspect of the invention there is provided a method of producing monoclonal antibodies according to the invention using hybridoma cell lines according to the invention.

In a further aspect of the invention there is provided a method for preparing a hybridoma cell-line producing monoclonal antibodies according to the invention comprising the steps of:
i) immunizing an immunocompetent mammal with an immunogen comprising at least one polypeptide having the amino acid sequence as represented in Table 1 by Genbank accession number, or fragments thereof or at least one polypeptide encoded by a nucleic acid molecule as represented in SEQ ID NO 1-452;
ii) fusing lymphocytes of the immunised immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

The production of monoclonal antibodies using hybridoma cells is well-known in the art. The methods used to produce monoclonal antibodies are disclosed by Kohler and Milstein in Nature 256, 495-497 (1975) and also by Donillard and Hoffman, “Basic Facts about Hybridomas” in Compendium of Immunology V.II ed. by Schwartz, 1981, which are incorporated by reference.

According to a further aspect of the invention there is provided a diagnostic assay for the determination of cancer in a subject comprising the steps of:
i) providing an isolated cell sample;
ii) contacting the sample in (i) with a binding agent(s) that bind to a nucleic acid molecule as represented by the nucleic acid sequence in SEQ ID NO 1-452;
iii) determining the expression of said nucleic acid molecule in said sample when compared to a normal matched control sample.

In a preferred embodiment of the invention said binding agent(s) is an oligonucleotide primer. Preferably said assay is a polymerase chain reaction.

In an alternative preferred embodiment of the invention said binding agent is an antibody that specifically binds a polypeptide encoded by a nucleic acid molecule as represented in SEQ ID NO 1-452, or a polypeptide variant comprising an amino acid sequence that varies from a reference amino acid sequence by addition, deletion or substitution of at least one amino acid residue.

In a preferred embodiment of the invention said cancer is prostate cancer.

According to a further aspect of the invention there is provided a kit comprising a binding agent specifically reactive with a nucleic acid molecule as represented by the nucleic acid sequence in SEQ ID NO 1-452, or an agent specifically reactive with a polypeptide comprising an amino acid sequence encoded by a nucleic acid molecule comprising a nucleic acid sequence as represented in SEQ ID NO 1-452.

In a preferred embodiment of the invention said kit further comprises an oligonucleotide or antibody specifically reactive with said nucleic acid molecule or said polypeptide.

Preferably said kit comprises a thermostable DNA polymerase and components required for conducting the amplification of nucleic acid. Preferably said kit includes a set of instructions for conducting said polymerase chain reaction and control nucleic acid.

In an alternative preferred embodiment of the invention said kit comprises an antibody specifically reactive with a polypeptide comprising an amino acid sequence encoded by a nucleic acid sequence as represented in SEQ ID NO 1-452.

Preferably said kit comprises components required for conducting an immunoassay including, for example, a secondary antibody specifically reactive with a primary antibody that specifically binds said polypeptide(s) and enzyme reagents required to detect the binding of said secondary antibody with said primary antibody.

According to a further aspect of the invention there is provided a method to screen for an agent that modulates the activity of a polypeptide encoded by a nucleic acid molecule selected from the group consisting of:
a) a nucleic acid molecule comprising a nucleic acid sequence as represented in SEQ ID NO 1-452;
a) a nucleic acid molecule consisting of a nucleic acid sequence as represented in Table 1 by Genbank accession number;
b) a nucleic acid molecule that hybridises under stringent hybridisation conditions to the nucleic acid molecule in (i) or (ii) above and which encodes a polypeptide wherein said polypeptide is stem cell specific;
i) forming a preparation comprising a polypeptide, or sequence variant thereof, and at least one agent to be tested;
ii) determining the activity of said agent with respect to the activity of said polypeptide.
In a preferred method of the invention said agent is an antagonist. In an alternative preferred method of the invention said agent is an agonist.

Agents identified by the screening method of the invention include, antibodies, siRNA, aptamers, small organic molecules, (for example peptides, cyclic peptides), dominant negative variants of the polypeptides herein disclosed.

As mentioned above, the invention also provides, in certain embodiments, “dominant negative” polypeptides derived from the polypeptides herein disclosed. A dominant negative polypeptide is an inactive variant of a protein, which, by interacting with the cellular machinery, displaces an active protein from its interaction with the cellular machinery or competes with the active protein, thereby reducing the effect of the active protein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to another transcription factor or to a promoter site in the control region of a gene but does not increase gene transcription can reduce the effect of a normal transcription factor by occupying promoter binding sites without increasing transcription.

It will be apparent to one skilled in the art that modification to the amino acid sequence of peptides agents could enhance the binding and/or stability of the peptide with respect to its target sequence. In addition, modification of the peptide may also increase the in vivo stability of the peptide thereby reducing the effective amount of peptide necessary to inhibit the activity of the polypeptides herein disclosed. This would advantageously reduce undesirable side effects which may result in vivo. Modifications include, by example and not by way of limitation, acetylation and amidation. Alternatively or preferably, said modification includes the use of modified amino acids in the production of recombinant or synthetic forms of peptides. It will be apparent to one skilled in the art that modified amino acids include, for example, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. Other modifications include amino acids with a C₂, C₃ or C₄ alkyl R group optionally substituted by 1, 2 or 3 substituents selected from halo (e.g. F, Br, I), hydroxy or C₁-C₄ alkoxy. It will also be apparent to one skilled in the art that peptides which retain p53 binding activity could be modified by cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363.

According to a further aspect of the invention there is provided a method to treat a subject for a cancer comprising administering an effective amount of an agent according to the invention.

In a preferred method of the invention said subject is human.

In a preferred method of the invention said cancer is prostate cancer.

According to a further aspect of the invention there is provided a method to immunize an animal against a cancerous condition comprising administering an effective amount of a nucleic acid or polypeptide encoded by a nucleic acid molecule selected from the group consisting of according to the invention.

In a preferred method of the invention said animal is a human.

In a further preferred embodiment of the invention said cancer is prostate cancer.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following Figures and Materials and Methods;

FIG. 1: Verification of CD133 as a stem cell marker of prostatic epithelia: 1A: A paraffin section of prostatic acini labelled with the nuclear stain DAPI (Blue) and anti CD133 directly conjugated to PE (Red). 1B: Basal cells with the phenotype α₂β₁^{hi}/CD133⁺ have a higher colony forming efficiency (CFE) than α₂β₁^{low}/CD133⁻. (CFE) was calculated as the number of colonies formed per number of selected cells ×100%. CFEs are expressed as the ratio of the control CFE. Results show means ±s.e.m of four experiments. 1C. Xenografts of prostate acini formed by transplantation of α₂β₁^{hi}/CD133⁺ basal cells stained with (A) Haematoxylin and Eosin, (13) 34βE12, (C) anti-K18, (D) anti-PAP (E) Anti-androgen receptor. Bar 40 μm;

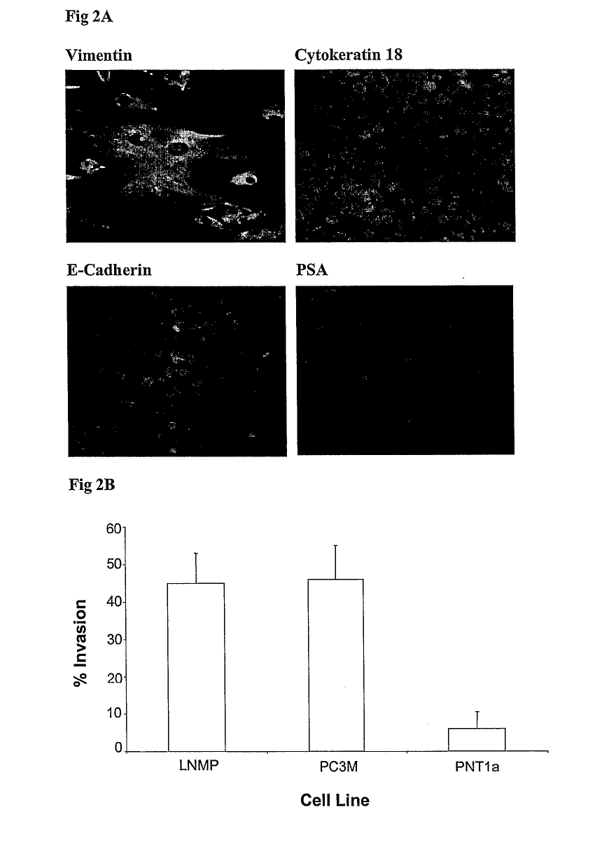

FIG. 2: Characterisation of tumour ‘stem’ cells from a lymph node metastasis of the prostate (LNMP). 2A. Tumour cells selected on the basis of □₂□₁/CD133 differentiate in culture. 2B. Invasion assay activity of LNMP in comparison to PC3M and an immortalised prostate epithelial cell line, PNT1a;

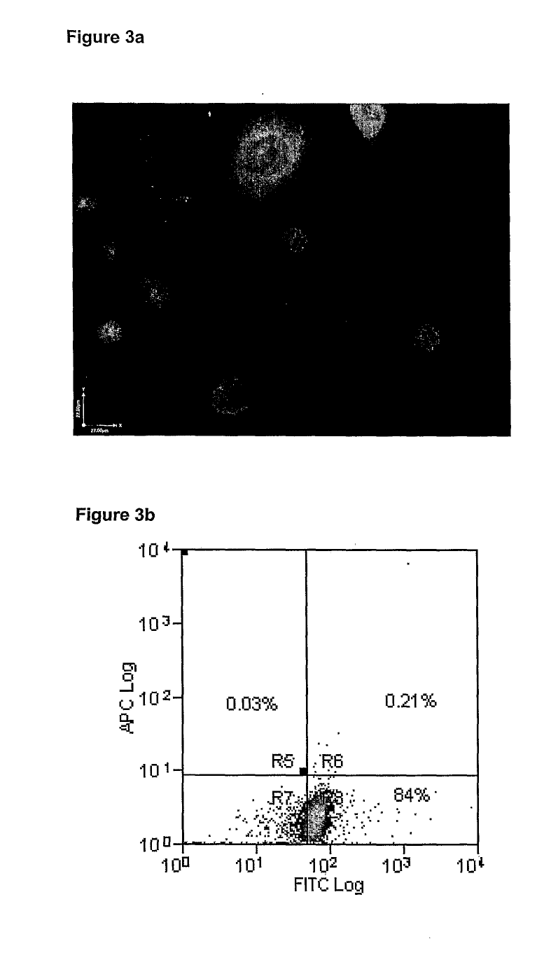

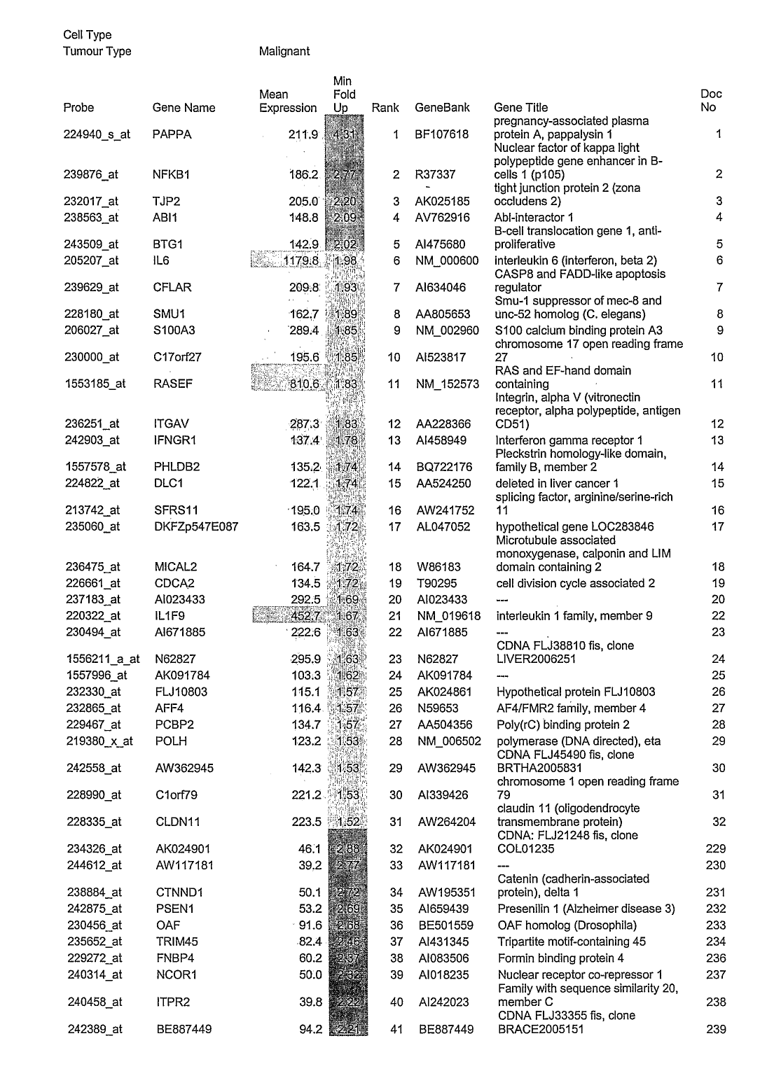

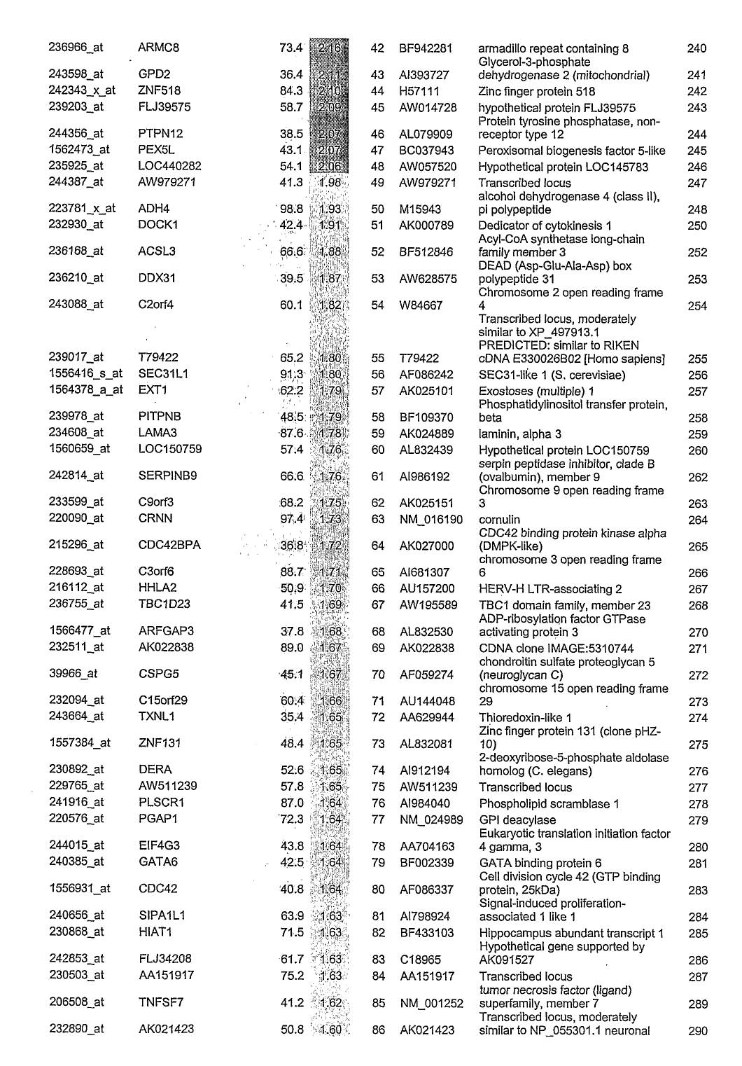

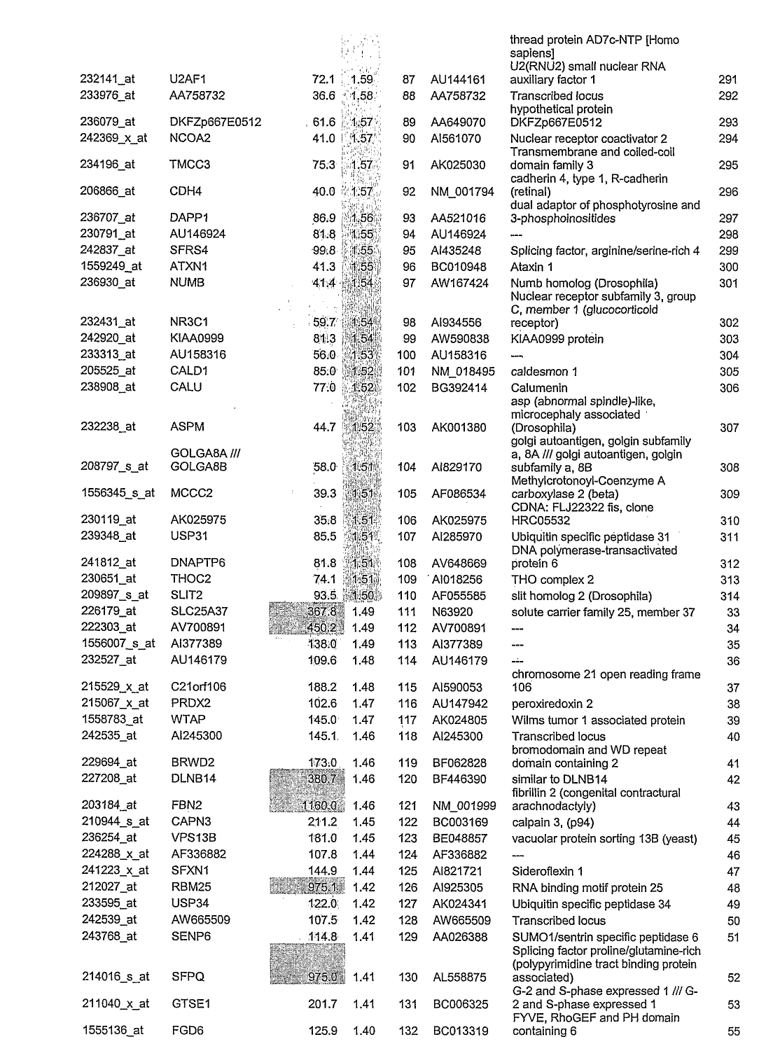

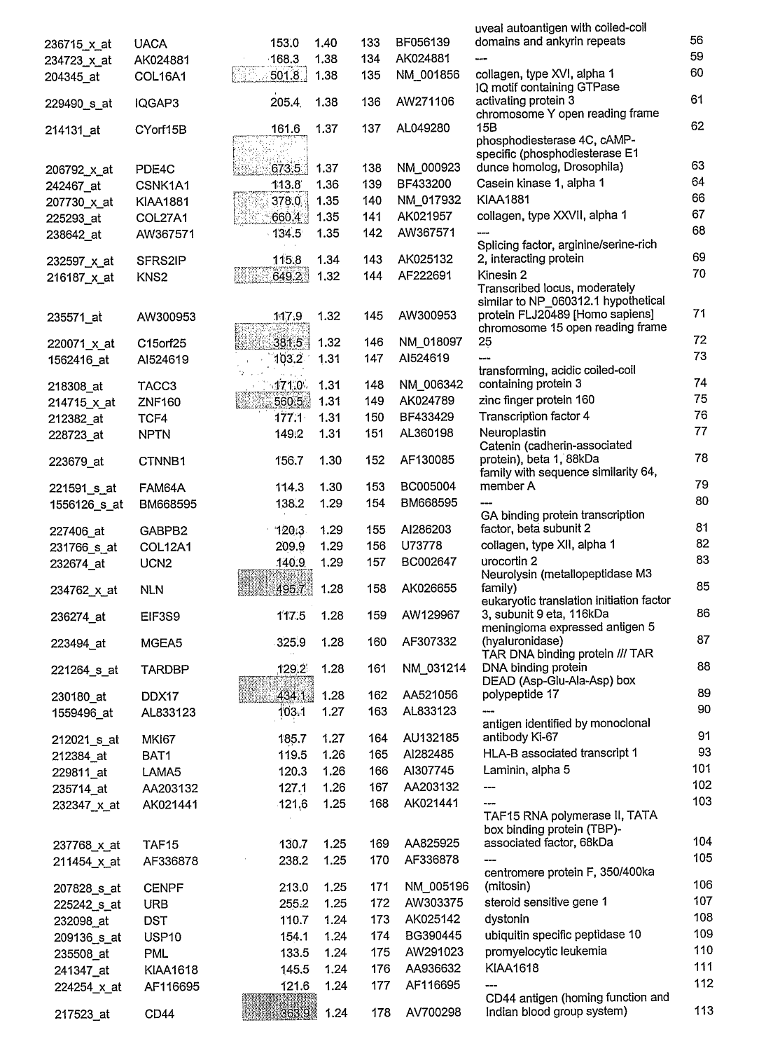

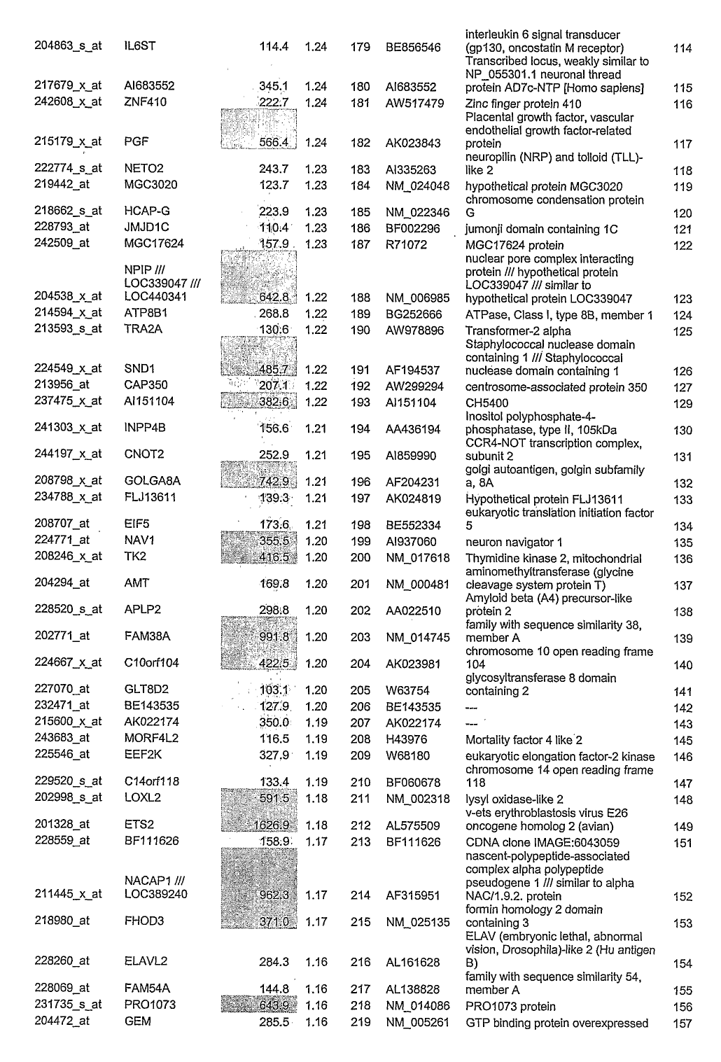

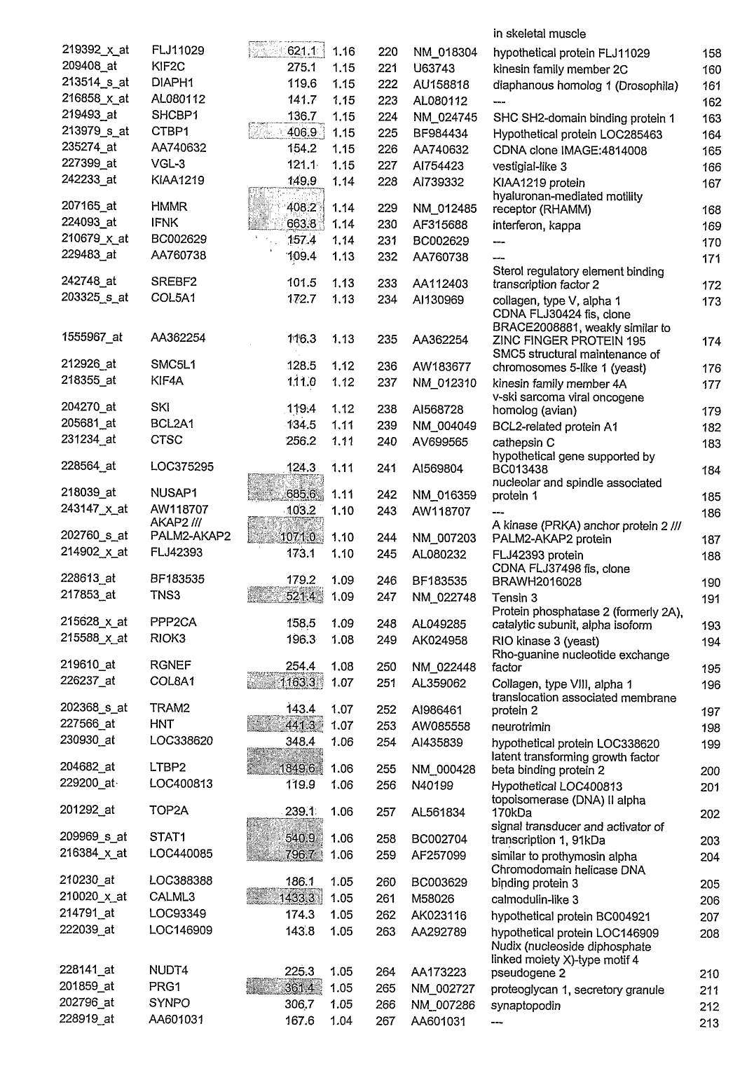

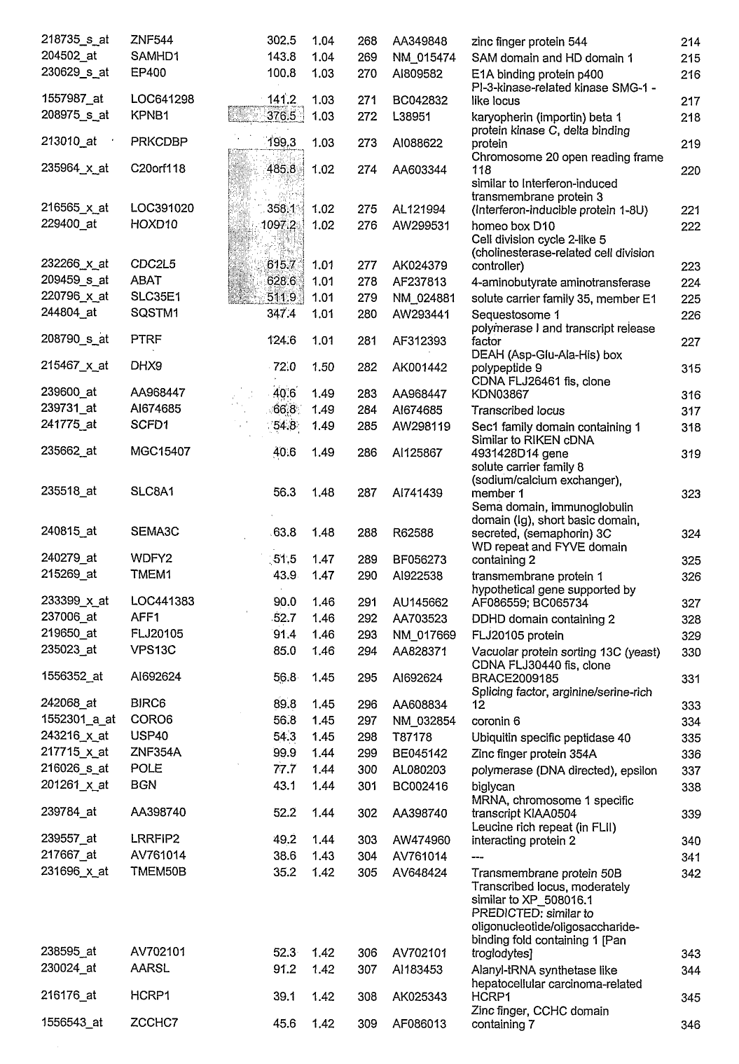

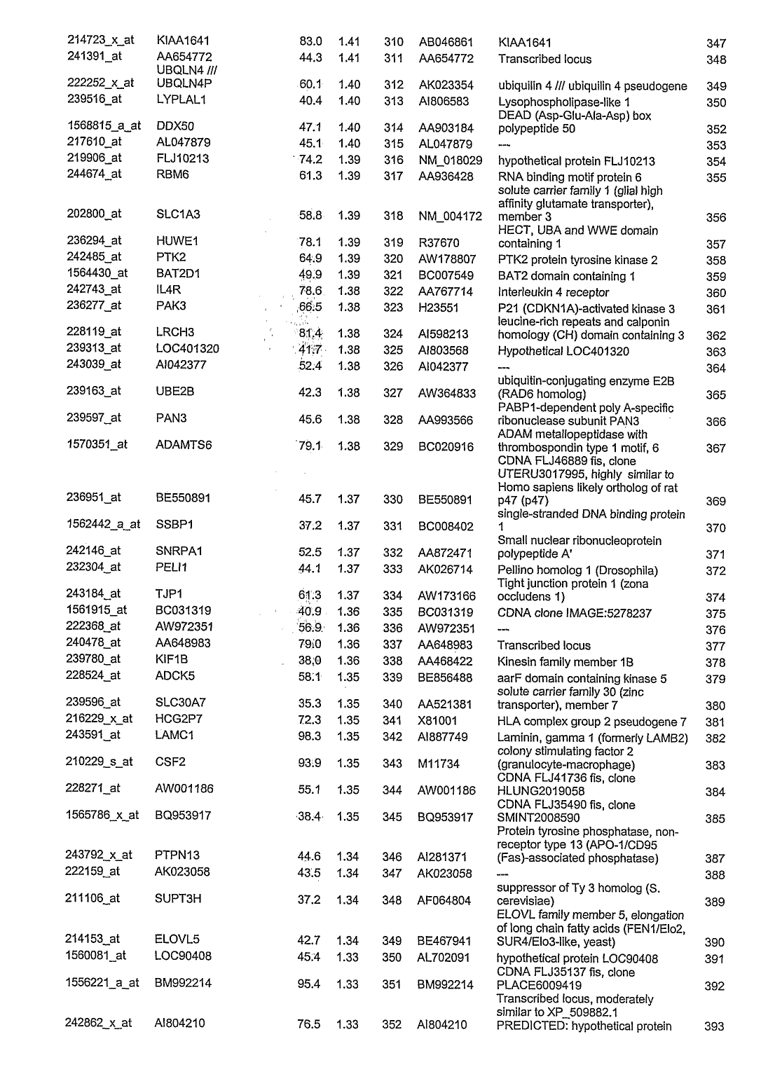

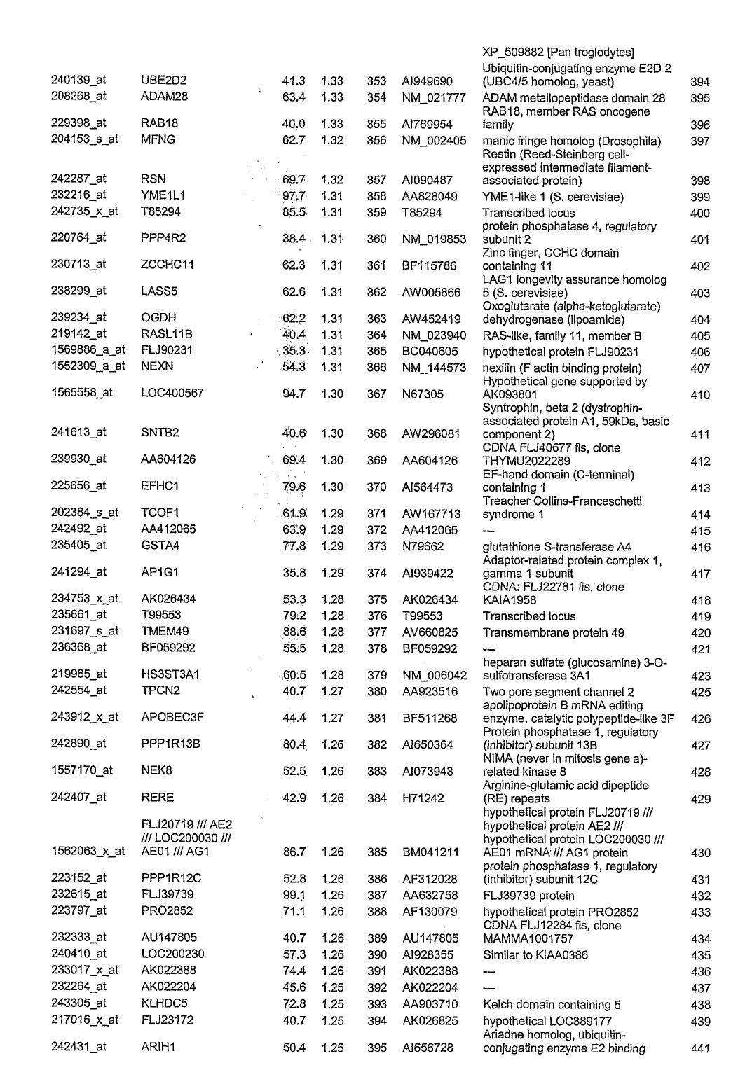

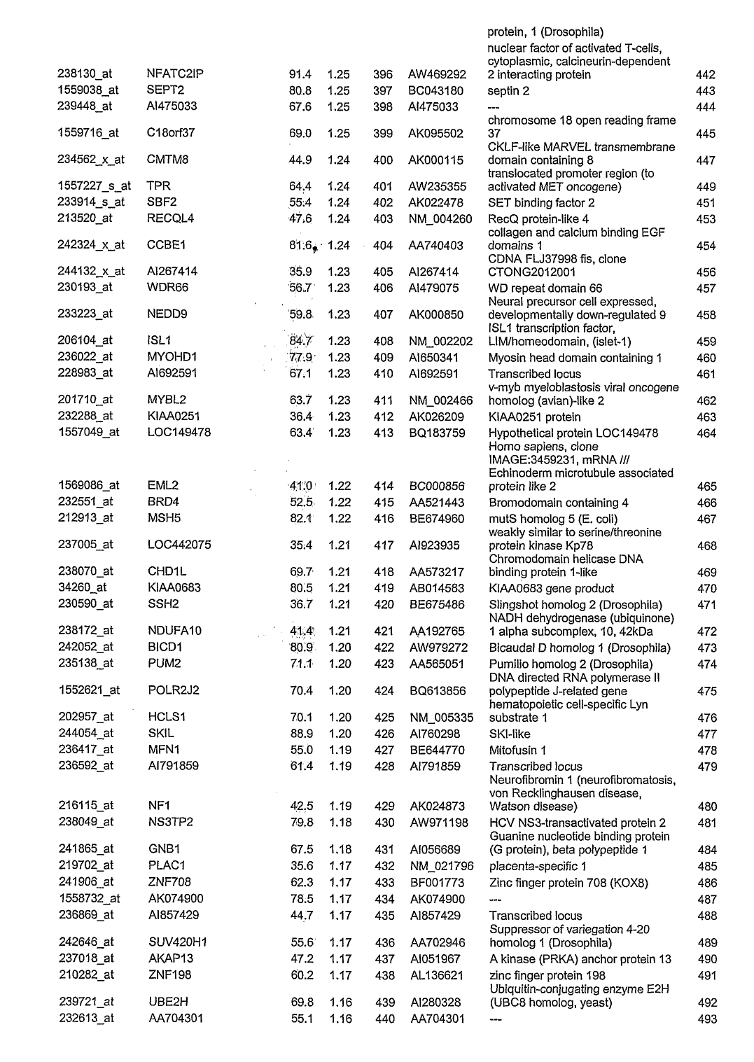

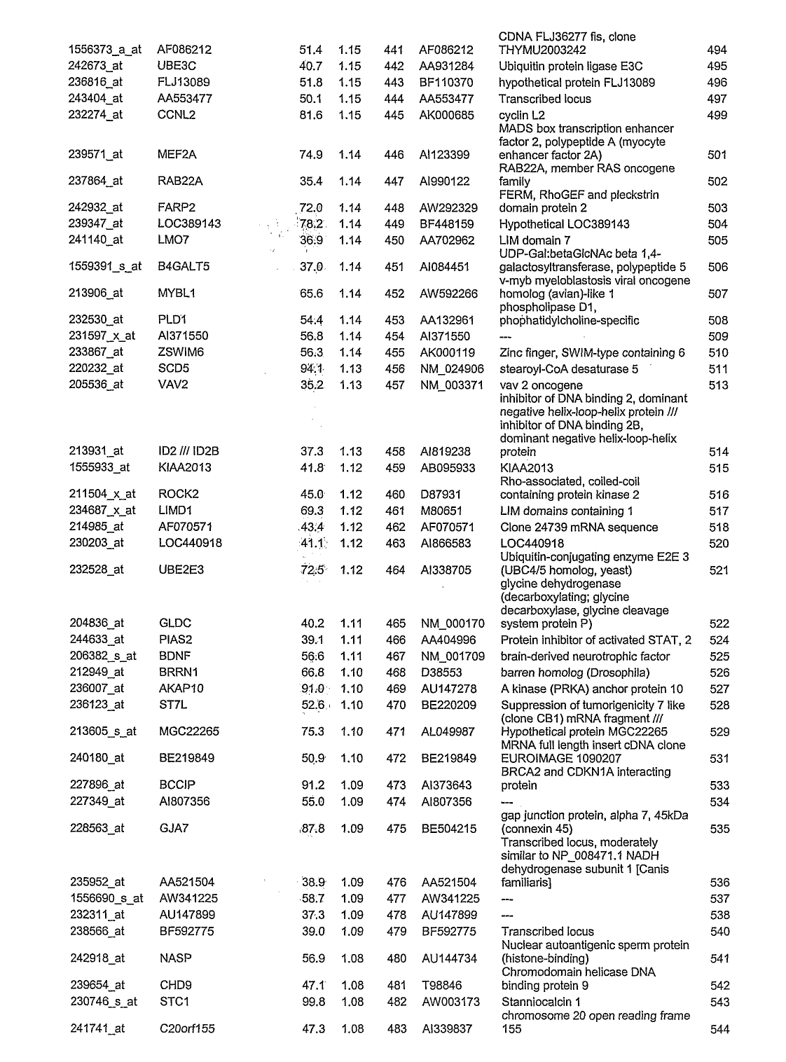

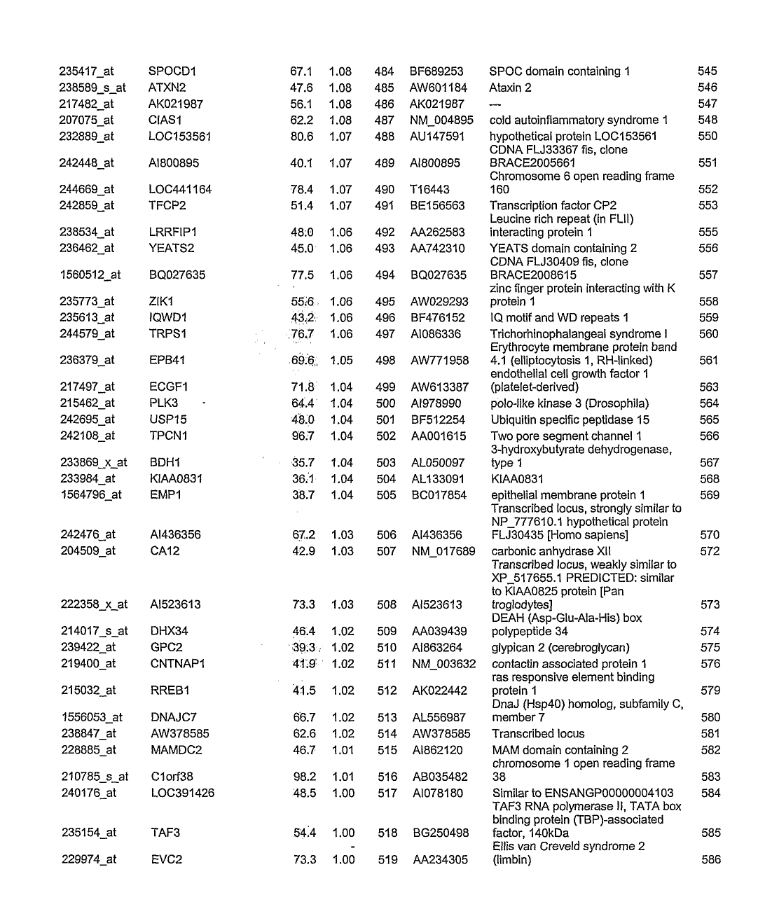

FIG. 3a represents NFkB Expression from α₂^{hi}/CD133+ cells (PE434 cells; Gleason 9); FIG. 3b represents a FACS dot plot of NFkB and CD133+ expression; 84% of cells are positive for NFkB alone and 0.21% are positive for NF3 and CD133.

Table 1 summarises array analysis of prostate stem cells and prostates cancer stem cells. Genes/nucleic acid and amino acid sequences are identified by Genbank accession number which can be accessed at http://www.ncbi.nlm.nih.gov. Genes are also identified by common name. The content of each accession entry is incorporated by reference and including the amino acid sequences of individual genes.

### Materials and Methods

### Genotype of Isolated Tumour Stem Cells

Using a combination of microsatellite markers associated with sporadic prostate cancer (8p 10q 16p) we can determine whether the isolated HEA⁺/CD44⁺/α₂β₁^{hi}/CD133⁺ cells display loss of heterozygosity patterns characteristic of prostate tumours in comparison to blood lymphocyte DNA from the same patient. The analysis is carried out on a microsampling of cultures with 3 MM paper and fluorescently labelled PCR primers (MacIntosh et al., 1998). This will enable us to discriminate between normal and cancer cells and determine whether stem cells are indeed targets for transforming events.

### Proliferative, Differentiative and Malignant Potential of Putative Cancer Stem Cells

Distinct populations of tumour cells are isolated and their proliferative, differentiative and malignant potential determined in vitro and in vivo. The following populations (HEA⁺/CD44⁻ (luminal cells), HEA⁺/CD44⁺ (basal cells), HEA⁺/CD44⁺/α₂β₁^{low}/CD133⁻ (transit cells), HEA⁺/CD44⁺/α₂β₁^{hi}/CD133⁺ (stem cells) are isolated and compared with the unsorted tumour population.

### Colony Forming Efficiency (CFE): Anchorage Independent and Anchorage-Dependent Growth

The transforming potential of distinct populations (as above) of cancer cells (anchorage independence) is measured by their ability to form colonies in soft agar. Individual colonies are counted after 21 days using an inverted microscope. Comparisons are made of CFE and colony size.

### Morphogenesis in Gels of Reconstituted Basement Membrane Matrix

We have determined the potential of tumour stem cells and their progenitors to undergo glandular morphogenesis in reconstituted basement membrane (e.g. Matrigel). We have demonstrated that normal basal cells can undergo glandular morphogenesis when grown in a collagen based matrix, (e.g. Matrigel) with stroma, in the presence of androgens. Spheroids are generated which are architecturally and phenotypically similar to in vivo acini and are often branched alveolar- and duct like (Lang et al., 2001). In contrast, cancer cells often form large aggregates of spindle-shaped cells with no obvious organisation. Nonetheless, the structures will often contain cells that show some degree of differentiation and can be compared to the original tumour.

### Invasion Assays

The ability of these stem cells to migrate across Matrigel is determined by the modified Boyden-chamber method (Albini et al., 1987). Migration rates will be evaluated using time-lapse confocal microscopy, using cells labelled with EGFP. We have generated prostate epithelium expressing low levels of EGFP. Recombinant retrovirus based on pLNCX-EGFP(2) generated will be used to infect the cell populations and G418 resistant colonies will be used in motility assays. The low levels of GFP expression will be used to track invasion and motility in real time.

### In Vivo Tumourigenesis

Tumour stem cells must possess key criteria that define normal stem cells: after transplantation they must proliferate, differentiate and self-renew. To determine the ability of distinct tumour phenotypes, to colonise in vivo, grafts of stem cells, transit cells, basal cells, luminal cells and unsorted cells are introduced into the prostates of 6 to 8 week old male, immuno-compromised mice. The mice are treated hormonally at the time of grafting by subcutaneous implantation of sustained release testosterone pellets. The number of cells from each population that successfully engraft and initiate tumour proliferation is determined by varying the number of cells implanted. The self-renewal capacity of the distinct populations is determined by transplanting serially into secondary recipients.

### Array Sample and Data Processing

### Total RNA Extraction

δ₂^{high}/CD133⁺ Cells

Total RNA is extracted from up to 1×10⁴ selected cells using QIAgen RNeasy micro columns. Cells are lysed in 100 μl RLT buffer+1% β-mercaptoethanol and the manufactures protocol for “total RNA isolation from animal cells” is followed (RNeasy_Micro0403.pdf, pages 39-44, which is incorporated by reference).

### α₂^{low} Cells

Total RNA is extracted from between 1×10⁵ and 1×10⁶ selected cells using QIAgen RNeasy mini columns. Cells are lysed in 350 μl RLT buffer+1% β-mercaptoethanol and the manufactures protocol for “isolation of total RNA from animal cells” cells is followed (RNeasy_Mini0601.pdf, pages 31-35, which is incorporated by reference).

RNA yields are determined spectrophotometrically at 260 nm and RNA integrity checked by capillary electrophoresis using an Agilent 2100 bioanalyzer.

Production of Fragmented Labelled cRNA

Total RNA is amplified using two rounds of cDNA synthesis and IVT (in vitro transcription) and biotin labelled by following the Affymetrix small scale labelling protocol vII (smallv2_technote.pdf which is incorporated by reference) with the following modifications:
1. 10-50 ng of total RNA is used per sample (step 1).
2. T4 DNA polymerase steps in the two second strand cDNA synthesis reactions are omitted (steps 2 & 7).
3. Second cycle, IVT for cRNA amplification and labelling (step 9) uses the Affymetrix GeneChip IVT labelling kit instead of the ENZO BioArray HighYield RNA transcript labelling kit and the Affymetrix eukaryotic sample and array processing standard protocol (expression_s2_manual-0604.pdf, section 2.1.34-2.1.35 which is incorporated by reference) is followed for this stage.

The quality of first and second round cRNA products and fragmented cRNA are checked by capillary electrophoresis using an Agilent 2100 bioanalyzer.

### Array Hybridisation

Labelled fragmented cRNA (15 μg) is hybridised to oligonucleotide probes on an Affymetrix HG-U133plus2 GeneChip. For hybridisation, washing, staining and scanning the Affymetrix eukaryotic sample and array processing standard protocol (expression_s2_manual_{—}0604.pdf, section 2.2.3-2.3.17 which is incorporated by reference) is followed.
1. Hybridisation is conducted using an Affymetrix Hybridisation Oven 640.
2. Washing and staining stages are conducted using an Affymetrix Fluidics Station 450 using the EukGE-WS2v5 protocol.
3. Scanning of arrays is done with an Affymetrix Gene Scanner 3000.

### Data Processing

Scanned GeneChip images are processed using Affymetrix GCOS software to derive an intensity value and flag (present, absent or marginal) for each probe. Probe intensities are derived using the MASS algorithm.

Comparisons between different sample datasets are conducted using Agilent GeneSpring GX software. Datasets to be compared are first normalised using three steps (consecutively applied in the order given):
1. Transform values <0.01 to 0.01
2. Normalise each chip to the 50 percentile of the measurements taken for that chip.
3. Normalise each probe to the median of the measurements for that probe.

For the purpose of analysing the data the following parameters are applied:
1. All cells derived from prostate cancer specimens are classed malignant, all cells derived from BPH specimens are classed benign (“tumour type” parameter)
2. All cells obtained by selection for high integrin α₂β₁ expression and CD133 are classed stem cells, all cells selected for low integrin α₂β₁ expression are classed committed basal cells (“cell type” parameter).
3. Further interpretations of the data allow for the combining of the above parameters to derive the conditions: malignant stem cells, malignant committed basal cells, benign stem cells and benign committed basal cells.

Low quality and uninformative data is removed using three selections (consecutively applied in the order given):
1. Remove probes flagged “absent” in all samples.
2. Remove probes with standard deviation within a parameter class of >1 in at least 3 of the 4 conditions.
3. Remove probes with less than a 2-fold overall change in normalised expression value between all four of the conditions.

### Stem Cell Markers

The invention relates to gene markers of stem cells, typically prostate stem cells, and in particular cancer stem cells, for example prostate cancer stem cells; therapeutic agents and diagnostic assays based on said stem cell genes and expression products; and including screening assays to identify therapeutic agents.

### BACKGROUND

A problem underlying the effective treatment of cancerous conditions is the identification of a population of cells in a tumour that have the ability of sustaining the growth of a tumour. The evidence suggests that tumours are clonal and are therefore derived from a single cell. However, there are few studies that identify and characterise those cells types that are responsible for maintaining tumour cell growth. Some have searched for these so called “cancer stem cells”.

We have identified CD133, which is expressed by primitive haematopoietic stem cells and developing epithelia as a further stem cell marker for prostate epithelia. CD133 cells are restricted to the α₂β₁^{hi} population (the receptor for type I collagen) and are located in the basal layer, often at the base of a budding region or branching point (FIG. 1A). α₂β₁^{hi}/CD133⁺ cells exhibit two important attributes of epithelial stem cells: they possess a high in vitro proliferative potential (FIG. 1B) and can reconstitute prostatic-like acini in immunocompromised male nude mice FIG. 1C).

In our co-pending application (WO 2005/089043) we describe the isolation of prostate stem cells which have been directly isolated from lymph node and prostate glands from a series of patient samples. These stem cells express markers that characterise the cells with stem cell properties. The following markers are typically expressed as prostate stem cell markers; human epithelial antigen (HEA), CD44, α₂β₁^{hi} and CD133. Morphologically the cells range from fibroblastoid (expressing high levels of vimentin which is typical of transformed cells) or epithelial, and are capable of producing progenitors associated with prostate epithelial differentiation. Invasion assays, using Matrigel-coated filters have determined that these cells have 2-3 fold greater capacity to invade through Matrigel than PC3M (a highly metastatic sub-line of PC3 cells).

### DESCRIPTION OF THE DISCLOSURE

We have conducted array analysis of CD133 positive prostate stem cells and compared expression of genes between CD133 positive stem cells and CD133 positive cancer stem cells.

### Growth Factors/Receptors

The array analysis has identified a number of genes that encode proteins that are either growth factors/receptors or proteins involved in signal transduction pathways that result in stimulation of cell growth and/or cell proliferation. A group of growth factors, referred to as cytokines, are involved in a number of diverse cellular functions. These include, by example and not by way of limitation, modulation of the immune system, regulation of energy metabolism and control of growth and development. Cytokines mediate their effects via receptors expressed at the cell surface on target cells. Cytokine receptors can be divided into three separate sub groups. Type 1 (e.g. growth hormone (GH) family) receptors are characterised by four conserved cysteine residues in the amino terminal part of their extracellular domain and the presence of a conserved Trp-Ser-Xaa-Trp-Ser motif in the C-terminal part. The repeated Cys motif is also present in Type 2 (interferon family) and Type III (tumour necrosis factor family).

A further group of growth factors are involved in angiogenesis. Angiogenesis is the development of new blood vessels from an existing vascular bed and is a complex multistep process that involves the degradation of components of the extracellular matrix and then the migration, cell-division and differentiation of endothelial cells to form tubules and eventually new vessels. Angiogenesis is involved in pathological conditions such as tumour cell growth. Genes involved in angiogenesis include, by example and not by way of limitation; vascular endothelial growth factor (VEGF, VEGF B, VEGF C, VEGF D); transforming growth factor (TGFb); acidic and basic fibroblast growth factor (aFGF and bFGF); and platelet derived growth factor (PDGF).

Many receptors for growth factors and the like are membrane bound via glycosylphosphatidylinositol (GPI). GPI-anchors are post-translational modifications to proteins that add glycosylphosphatidylinositol which enable these proteins to anchor to the extracellular side of cell membranes. Typically, extracellular proteins which have a GPI anchor do not have transmembrane or cytoplasmic domains. GPI anchor proteins occur in all eukaryotes and form a diverse variety of proteins that includes, for example, membrane associated enzymes and adhesion molecules.

Genes that are involved in signal transduction are often associated with cell membrane localised receptors which upon activation results in a signal transduction cascade leading to activation of transcription and cell proliferation. For example, the PTEN gene product is a multiple-specificity phosphatase that can antagonise phosphoinositide lipid kinases (hereinafter PI3K) by degrading phosphoinositide (PI) (3,4,5) P3 back to PI(4,5)P2 in addition to its ability to dephosphorylate a range of protein targets such as focal adhesion kinase (FAK). Phosphoinositides have been implicated in a variety of cellular processes as diverse as vacuolar protein sorting, cytoskeletal remodelling and mediating intracellular signalling events through which growth factors, hormones and neurotransmitters exert their physiological effects on cellular activity, proliferation and differentiation.

### Extracellular Matrix Associated

The array analysis has identified a number of genes that encode proteins that are extracellular matrix proteins. The Extracellular Matrix (ECM) is a complex mixture of non-living material which surrounds cells in multicellular organisms. In vertebrates the ECM comprises a mixture of protein and carbohydrate (and minerals in the example of bone). The protein component comprises proteins and glycoproteins (proteins that are modified by the addition of sugar moieties) such as collagens (collagens I-XII); laminins which are found in the basal lamina a structure to which epithelial cells associate; fibronectin which functions to bind cells to the ECM; and elastins which provide skin with the flexibility. Proteoglycans are also found in the ECM and these glycoproteins comprise more carbohydrate than protein. Several sugars are added to proteoglycans the most abundant of which is acetylglucosamine. Many proteoglycans are sulphated, for example chondroitin sulphate, heparan sulphate, keratin sulphate and hyaluronic acid.

Most vertebrate cells cannot survive unless associated with the ECM and the loss of anchorage to the ECM is often associated with cell transformation in cancer. Cells attach to the ECM via anchoring molecules expressed by the cell called intergrins which bind the ECM via collagen, laminins and fibronectin. Integrins are cell membrane proteins that bind the extracellular matrix via their extracellular domain which projects from the cell surface. The integrin intracellular domain contacts the actin filaments of the cytoskeleton. In cancer metastasis the primary cancer cell becomes motile and is able to metastase (transfer) to other tissues to form secondary cancers. It is the secondary cancer that eventually kills the subject. In order to metastase the cancer cell has to penetrate the basement membrane of the tissue to gain access to the blood and lymph systems which transports the cancer cell around the body. The degradation of the ECM around a tumour is mainly catalysed by metalloproteases (MMP) which are secreted by stromal cells that surround the tumour. There is increasing evidence that cancer cells stimulate MMP production by fibroblasts by the paracrine secretion of hormones.

MMP's are an expanding group of proteases that can be classified into 3 groups. Group 1 includes collagenases that degrade connective tissue collagen, for example MMP-1, MMP-8 and MMP-13; group 2 includes gelatinases that degrade basement membrane collagens and include MMP-2 and MMP-9; a third group includes the stromelysins that degrade ECM proteoglycans, laminin and fibronectin and include MMP-10 and MMP-11. The activity of MMP's can be enhanced by pro-inflammatory cytokines such as IL-1 and TNFα.

### Transcription Factors/Transcription Related

The array analysis has identified a number of genes that encode proteins that are transcription factor proteins or proteins that are related to transcription.

Transcription factors are proteins that bind to DNA enhancer or promoter elements. Often these are near to the start of transcription of a gene. Transcription factors either inhibit or facilitate RNA polymerase transcription initiation and also the maintenance of an active transcription complex. Transcription factors contain two basic functional domains. A transactivation domain which is a region of the protein which interacts with other parts of the transcription machinery, for example the RNA polymerase or other transcription factors; and a DNA binding domain which comprises amino acids which recognise specific bases within the promoter region of the gene. In some examples enhancer elements can be positioned at a distance from the start of transcription or even within introns, Lewin B, 1994. Genes V, Oxford University Press, Oxford. Frequently DNA binding domains interact with nucleotide sequences or motifs which are sites to which the transcription factor binds to enhance or repress transcription.

An important family of the transcription factors comprises the homeodomain proteins. This family of transcription factors is characterised by the so-called homeodomain region which consists of 16 amino acids arranged in a helix-turn-helix conformation. Some transcription factors have both a homeodomain and a second DNA-binding region. In some examples, the region that comprises the homeodomain and the second DNA-binding region is called the POU domain.

A further example of a family of transcription factors which contain a conserved binding domain is the helix-loop-helix domain. The muscle specific transcription factor MyoD contains this motif, as do several D. melanogaster proteins that determine the cell fate in the D. melanogaster peripheral nervous system.

A related family of transcription factors are referred to as the basic leucine zipper transcription factor family or bZip. The bZip proteins are dimers, each of whose subunits contain a basic DNA-binding domain at the carboxyl end followed closely by a helix containing several leucine residues. Examples of bZip family members are C/EBP, Ap1, and the yeast transcription factor GCN4

A large group of transcription factors are the nuclear hormone receptors. It is known that steroid hormones increase the transcription of specific groups of genes. Once the hormone has entered the cell, it binds to its specific receptor protein, converting that receptor into a conformation that is able to enter the nucleus and bind specific DNA sequence motifs. The family of steroid hormone receptors includes proteins that recognise oestrogen, progesterone, testosterone and cortisone as well as non-steroid lipids such as retinoic acid, thyroxine and vitamin D.

It is known that the way in which DNA is packaged as chromatin can influence the expression of genes. There are several levels of structural packaging of DNA leading from a double stranded helix to a mitotic chromosome, after which the DNA is some 50,000 times shorter than its extended length. Double-stranded helical DNA is wound around the structural unit of a nucleosome, comprising an octamer core composed of 4 types of histones: two each of the H2A, H2B, H3, and H4 proteins. Approximately 166 base pairs are bound to the nucleosome through electrostatic forces between the negatively charged phosphate groups in the DNA backbone and positively charged amino acids (e.g., lysine and arginine) in the histone proteins.

Nucleosomes are organised into the next structural level of the chromatin fibre, also referred to as a solenoid. Chromatin structure is not static and the regulated alteration in structure is termed ‘chromatin remodelling’. This process has been defined as any event that alters the nuclease sensitivity of a region of chromatin, and can occur independently or in concert with processes such as transcription.

Reversible acetylation of evolutionary conserved lysine residues in core histone proteins plays a critical role in transcriptional regulation, cell cycle progression, and developmental events. The steady state of histone acetylation is controlled by the enzymatic activities of multiple histone acetyltransferases (HATs) and histone deacetylases (HDACs). Histone hyperacetylation is associated with transcriptional activity while histone hypoacetylation correlates with transcriptional quiescence and so histone deacetylases can be considered as enzymatic transcriptional repressors.

In general, histone deacetylases do not target genes directly through specific DNA-binding sites. Rather, deacetylases are localized to genes targeted for repression as part of a protein complex. Other proteins that are part of this complex, termed co-repressors, are responsible for targeting the genes to be repressed. In humans, four highly homologous class I HDAC enzymes (HDAC1, HDAC2, HDAC3, and HDAC8) have been identified to date, with HDAC1, HDAC2 and HDAC3 being ubiquitously expressed in many different cell types (Yang et al., 1997 and 2002). HDAC1 and HDAC2 are the human orthologues of the yeast transcriptional regulator RPD3. Analysis of the predicted amino acid sequence of HDAC3 revealed an open reading frame of 428 amino acids with a predicted molecular mass of 49 kDa.

### DNA Replication and Repair

The array analysis has identified a number of genes that encode proteins that are involved in DNA replication or repair of DNA. DNA damage can occur through a number of agents. For example, certain wavelengths of radiation, (e.g. gamma rays or X-rays), ultraviolet rays especially UV-C rays that are absorbed strongly by DNA; highly reactive oxygen radicals produced during respiration and other metabolic processes; and chemical mutagens found in the environment which may be man made or naturally occurring.

DNA can be damaged in different ways. For example, the four bases that form DNA can be covalently modified at various positions; deamination of an amino group is a common modification resulting in a mutation of cytosine to uracil. Other modifications include mismatches, for example the conversion of thymidine to uracil, single strand breaks in the phosphate backbone of the DNA molecule and covalent crosslinks between bases which may be intra-strand or inter-strand. Several chemotherapeutic agents used in the treatment of cancer act as crosslinking agents.

The Human Genome Project which has deduced the complete DNA sequence of human DNA has identified around 130 genes thought to be involved in DNA repair and replication. Damaged or inappropriate incorporation of bases can be corrected via several mechanisms. These include direct chemical reversal or excision repair. Excision repair results in removal of the damaged base and replacement with the correct base. There are three mechanisms of excision repair utilised by cells to repair DNA damage.

Base excision repair involves the removal of the damaged base by a DNA glycosylase; removal of its deoxyribose phosphate to produce gapped DNA; replacement of the correct nucleotide by a DNA polymerase β and ligation of the strand break by a DNA ligase.

Nucleotide excision repair involves recognition of the error by one or more protein factors; separating the DNA strands to produce a “bubble” by an enzyme called transcription factor 11H; scission at the 5′ and 3′ sides of the damaged area; replacement synthesis of the damaged area by DNA polymerases ε and σ; and ligation of the strand break by a DNA ligase.

Mismatch repair corrects mismatches in normal bases. The correction of mismatches utilises enzymes involved in base excision repair and proteins that recognise the mismatch for example proteins encoded by MSH2 and scission around the mismatch by MLH 1 and other proteins. A mutation in either of these genes has been associated with an inherited form of colon cancer. The repair of the mismatch is completed by the DNA polymerases ε and σ.

In addition the repair of single and double strand breaks in DNA also involves a number of proteins. The repair of single strand breaks utilises many of the proteins involved in base excision repair. A double strand break is repaired either by direct ligation of the free ends of the break or by homologous recombination. Errors in direct ligation are associated with certain cancers, for example Burkitt's lymphoma and B-cell leukaemia. The BRCA-1 and BRCA-2 genes function in homologous recombination and mutations in these genes are associated with breast and ovarian cancer.

We have conducted gene array analysis to identify genes that are characteristic of cancer stem cells which show an up regulation when compared to control stem cell samples from normal or benign stem cell populations. We herein disclose these genes and their use in the identification of therapeutic agents useful in the treatment of cancer, in particular prostate cancer, and in the development of diagnostic assays for the detection of the early on set of tumour cell growth. The present disclosure relates to the identification of cancer stem cell specific genes.

### STATEMENTS OF INVENTION

According to a first aspect of the invention there is provided agent that modulates the activity of a cancer stem cell specific nucleic acid molecule, or a polypeptide encoded by a cancer stem nucleic acid molecule, wherein said cancer cell specific nucleic acid molecule is selected from the group consisting of:
i) a nucleic acid molecule consisting of a nucleic acid sequence as represented in SEQ ID NO: 1-452;
ii) a nucleic acid molecule consisting of a nucleic acid sequence as represented in Table 1 by Genbank accession number;
iii) a nucleic acid molecule that hybridises under stringent hybridisation conditions to the nucleic acid molecule in (i) or (ii) above and which encodes a polypeptide wherein said polypeptide is stem cell specific, characterised in that said agent is for use as a pharmaceutical.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology-Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Allows Sequences that Share at Least 80% Identity to Hybridize)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Allows Sequences that Share at Least 50% Identity to Hybridize)

Hybridization: 6×SSC at RT to 55° C. for 16-20 hours

Wash at least twice: 2×-3×SSC at RT to 55° C. for 20-30 minutes each.

In a preferred embodiment of the invention said agent is an antagonist. Alternatively, said agent is an agonist.

According to a further aspect of the invention there is provided an agent that modulates the activity of a polypeptide comprising an amino acid sequence encoded by a nucleic acid molecule selected from the group consisting of:
i) a nucleic acid molecule as represented in SEQ ID NO: 1-452,
ii) a nucleic acid molecule that encodes a variant polypeptide wherein said variant polypeptide is modified by addition, deletion or substitution of at least one amino acid residue of the amino acid sequence encoded by a nucleic acid sequence selected from the group consisting of SEQ ID NO: 1-452 wherein said polypeptide is stem cell specific;
iii) a nucleic acid molecule that encodes a polypeptide consisting of an amino acid sequence as represented in Table 1 by Genbank accession number; characterised in that said agent is for use as a pharmaceutical.

A variant polypeptide may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan.

In addition, the invention features polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequence illustrated herein.

In a preferred embodiment of the invention said agent is a polypeptide. Preferably said polypeptide is an antibody or an active binding part of an antibody.

In a preferred embodiment of the invention said antibody is a monoclonal antibody or active binding part thereof.

In a preferred embodiment of the invention said antibody is a chimeric antibody or a humanised antibody produced by recombinant methods to contain the variable region of said antibody with an invariant or constant region of a human antibody.

Chimeric antibodies are recombinant antibodies in which all of the V-regions of a mouse or rat antibody are combined with human antibody C-regions. Humanised antibodies are recombinant hybrid antibodies which fuse the complimentarily determining regions from a rodent antibody V-region with the framework regions from the human antibody V-regions. The complimentarity determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen. Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not illicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In a preferred embodiment of the invention said agent is an antibody fragment.

Various fragments of immunoglobulin or antibodies are known in the art, i.e., Fab, Fab₂, F(ab′)₂, Fv, Fc, Fd, scFvs, etc. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact immunoglobulin molecule. A Fab₂ fragment comprises two joined Fab fragments. When these two fragments are joined by the immunoglobulin hinge region, a F(ab′)₂ fragment results. An Fv fragment is multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain variable region, or a fragment thereof containing the three CDRs of the heavy chain variable region, without an associated light chain moiety; and multi specific antibodies formed from antibody fragments, this has for example been described in U.S. Pat. No. 6,248,516. Fv fragments or single region (domain) fragments are typically generated by expression in host cell lines of the relevant identified regions. These and other immunoglobulin or antibody fragments are within the scope of the invention and are described in standard immunology textbooks such as Paul, Fundamental Immunology or Janeway et al. Immunobiology (cited above). Molecular biology now allows direct synthesis (via expression in cells or chemically) of these fragments, as well as synthesis of combinations thereof.

It is possible to create single variable regions, so called single chain antibody variable region fragments (scFv's). If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's. Alternatively said fragments are “domain antibody fragments”. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety.

In a preferred method of the invention said antibody fragment is a single chain antibody variable region fragment.

A fragment of an antibody or immunoglobulin can also have bispecific function binding two different epitopes of two different antigens.

Preferably said chimeric/humanised monoclonal antibody to said polypeptide is produced as a fusion polypeptide in an expression vector suitably adapted for transfection or transformation of prokaryotic or eukaryotic cells.

In a further preferred embodiment of the invention said antibodies are opsonic antibodies.

Phagocytosis is mediated by macrophages and polymorphic leukocytes and involves the ingestion and digestion of micro-organisms, damaged or dead cells, cell debris, insoluble particles and activated clotting factors. Opsonins are agents which facilitate the phagocytosis of the above foreign bodies. Opsonic antibodies are therefore antibodies which provide the same function. Examples of opsonins are the Fc portion of an antibody or compliment C3.

Preferably, said antibody is provided with a marker including a conventional label or tag, for example a radioactive and/or fluorescent and/or epitope label or tag.

In an alternative preferred embodiment of the invention said antibody, or antibody fragment had associated therewith or crosslinked thereto a therapeutic agent. Preferably said therapeutic agent is a chemotherapeutic agent.

Preferably said agent is selected from the group consisting of: cisplatin; carboplatin; cyclosphosphamide; meiphalan; cannusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; and camptothecin.

In an alternative preferred embodiment of the invention said agent is a nucleic acid molecule. For example, an antisense nucleic acid; an aptamer; or a small interfering RNA.

In a preferred embodiment of the invention said nucleic acid molecule is a small interfering RNA.

A technique to specifically ablate gene function is through the introduction of double stranded RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically derived from exons of the gene which is to be ablated.

The mechanism of RNA interference is being elucidated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA.

According to a further aspect of the invention there is provided a pharmaceutical composition comprising an agent according to the invention.

According to a further aspect of the invention there is provided a composition comprising a nucleic acid molecule selected from the group consisting of:
i) a nucleic acid molecule consisting of a nucleic acid sequence as represented in SEQ ID NO: 1-452;
ii) a nucleic acid molecule consisting of a nucleic acid sequence as represented in Table 1 by Genbank accession number;
iii) a nucleic acid molecule that hybridises under stringent hybridisation conditions to the nucleic acid molecule in (i) or (ii) above and which encodes a polypeptide wherein said polypeptide is stem cell specific.

According to a further aspect of the invention there is provided a composition comprising a polypeptide comprising an amino acid sequence selected from the group consisting of:
i) a polypeptide comprising an amino acid sequence as represented in Table 1 by Genbank accession number, or a variant polypeptide wherein said variant is modified by addition, deletion or substitution of at least one amino acid residue of the amino acid sequence presented in Table 1 by Genbank accession number;
ii) a polypeptide comprising an amino acid sequence encoded by a nucleic acid molecule comprising a nucleic acid sequence as represented in SEQ ID NO 1-452;
iii) a polypeptide comprising an amino acid sequence encoded by a nucleic acid molecule that hybridises under stringent hybridisation conditions to the nucleic acid molecule in (ii) above and which encodes a polypeptide wherein said polypeptide is stem cell specific wherein said composition is for use as a vaccine.

In a preferred embodiment of the invention said composition includes an adjuvant and/or a carrier.

An adjuvant is a substance or procedure that augments specific immune responses to antigens by modulating the activity of immune cells. Examples of adjuvants include, by example only, Freunds adjuvant, muramyl dipeptides, liposomes. A carrier is an immunogenic molecule which, when bound to a second molecule, augments immune responses to the latter. Some antigens are not intrinsically immunogenic yet may be capable of generating antibody responses when associated with a foreign protein molecule such as keyhole-limpet haemocyanin or tetanus toxoid. Such antigens contain B-cell epitopes but no T cell epitopes. The protein moiety of such a conjugate (the “carrier” protein) provides T-cell epitopes which stimulate helper T-cells that in turn stimulate antigen-specific B-cells to differentiate into plasma cells and produce antibody against the antigen. Helper T-cells can also stimulate other immune cells such as cytotoxic T-cells, and a carrier can fulfil an analogous role in generating cell-mediated immunity as well as antibodies.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents (for example, cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; and camptothecin.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing antibody aerosols without resort to undue experimentation.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of antibody or nucleic acid for producing the desired response in a unit of weight or volume suitable for administration to a patient. The response can, for example, be measured by determining the signal transduction enhanced or inhibited by the composition via a reporter system, by measuring downstream effects such as gene expression, or by measuring the physiological effects of the composition. Likewise, the effects of antisense/siRNA molecules can be readily-determined by measuring expression of the individual genes in cells to which an antisense/siRNA composition is added. Other assays will be known to one of ordinary skill in the art and can be employed for measuring the level of the response.

The doses of antibody or nucleic acid administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of antibody are formulated and administered in doses between 1 ng and 1 mg, and preferably between 10 ng and 100 μg, according to any standard procedure in the art. Where nucleic acids or variants thereof are employed, doses of between 1 ng and 0.1 mg generally will be formulated and administered according to standard procedures. Other protocols for the administration of the compositions will be known to one of ordinary skill in the art, in which the dose amount, schedule of injections, sites of injections, mode of administration (e.g., intra-bone) and the like vary from the foregoing. Administration of the compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Pharmaceutical compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation of antibody or nucleic acids, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a vector which is adapted for the expression of the humanised or chimeric antibodies according to the invention.

According to an aspect of the invention there is provided a cell which has been transformed or transfected with the vector encoding the humanised or chimeric antibody according to the invention

According to a further aspect of the invention there is provided a method for the production of the humanised or chimeric antibody according to the invention comprising:
(i) providing a cell transformed or transfected with a vector which comprises a nucleic acid molecule encoding the humanised or chimeric antibody according to the invention;
(ii) growing said cell in conditions conducive to the manufacture of said antibody; and
(iii) purifying said antibody from said cell, or its growth environment.

In a yet further aspect of the invention there is provided a hybridoma cell line which produces a monoclonal antibody as hereinbefore described.

In a further aspect of the invention there is provided a method of producing monoclonal antibodies according to the invention using hybridoma cell lines according to the invention.

In a further aspect of the invention there is provided a method for preparing a hybridoma cell-line producing monoclonal antibodies according to the invention comprising the steps of:
i) immunizing an immunocompetent mammal with an immunogen comprising at least one polypeptide having the amino acid sequence as represented in Table 1 by Genbank accession number, or fragments thereof or at least one polypeptide encoded by a nucleic acid molecule as represented in SEQ ID NO 1-452;
ii) fusing lymphocytes of the immunised immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

The production of monoclonal antibodies using hybridoma cells is well-known in the art. The methods used to produce monoclonal antibodies are disclosed by Kohler and Milstein in Nature 256, 495-497 (1975) and also by Donillard and Hoffman, “Basic Facts about Hybridomas” in Compendium of Immunology V.II ed. by Schwartz, 1981, which are incorporated by reference.

According to a further aspect of the invention there is provided a diagnostic assay for the determination of cancer in a subject comprising the steps of:
i) providing an isolated cell sample;
ii) contacting the sample in (i) with a binding agent(s) that bind to a nucleic acid molecule as represented by the nucleic acid sequence in SEQ ID NO 1-452;
iii) determining the expression of said nucleic acid molecule in said sample when compared to a normal matched control sample.

In a preferred embodiment of the invention said binding agent(s) is an oligonucleotide primer. Preferably said assay is a polymerase chain reaction.

In an alternative preferred embodiment of the invention said binding agent is an antibody that specifically binds a polypeptide encoded by a nucleic acid molecule as represented in SEQ ID NO 1-452, or a polypeptide variant comprising an amino acid sequence that varies from a reference amino acid sequence by addition, deletion or substitution of at least one amino acid residue.

In a preferred embodiment of the invention said cancer is prostate cancer.

According to a further aspect of the invention there is provided a kit comprising a binding agent specifically reactive with a nucleic acid molecule as represented by the nucleic acid sequence in SEQ ID NO 1-452, or an agent specifically reactive with a polypeptide comprising an amino acid sequence encoded by a nucleic acid molecule comprising a nucleic acid sequence as represented in SEQ ID NO 1-452.

In a preferred embodiment of the invention said kit further comprises an oligonucleotide or antibody specifically reactive with said nucleic acid molecule or said polypeptide.

Preferably said kit comprises a thermostable DNA polymerase and components required for conducting the amplification of nucleic acid. Preferably said kit includes a set of instructions for conducting said polymerase chain reaction and control nucleic acid.

In an alternative preferred embodiment of the invention said kit comprises an antibody specifically reactive with a polypeptide comprising an amino acid sequence encoded by a nucleic acid sequence as represented in SEQ ID NO 1-452.

Preferably said kit comprises components required for conducting an immunoassay including, for example, a secondary antibody specifically reactive with a primary antibody that specifically binds said polypeptide(s) and enzyme reagents required to detect the binding of said secondary antibody with said primary antibody.

According to a further aspect of the invention there is provided a method to screen for an agent that modulates the activity of a polypeptide encoded by a nucleic acid molecule selected from the group consisting of:
a) a nucleic acid molecule comprising a nucleic acid sequence as represented in SEQ ID NO 1-452;
a) a nucleic acid molecule consisting of a nucleic acid sequence as represented in Table 1 by Genbank accession number;
b) a nucleic acid molecule that hybridises under stringent hybridisation conditions to the nucleic acid molecule in (i) or (ii) above and which encodes a polypeptide wherein said polypeptide is stem cell specific;
i) forming a preparation comprising a polypeptide, or sequence variant thereof, and at least one agent to be tested;
ii) determining the activity of said agent with respect to the activity of said polypeptide.

In a preferred method of the invention said agent is an antagonist. In an alternative preferred method of the invention said agent is an agonist.

Agents identified by the screening method of the invention include, antibodies, siRNA, aptamers, small organic molecules, (for example peptides, cyclic peptides), dominant negative variants of the polypeptides herein disclosed.

As mentioned above, the invention also provides, in certain embodiments, “dominant negative” polypeptides derived from the polypeptides herein disclosed. A dominant negative polypeptide is an inactive variant of a protein, which, by interacting with the cellular machinery, displaces an active protein from its interaction with the cellular machinery or competes with the active protein, thereby reducing the effect of the active protein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to another transcription factor or to a promoter site in the control region of a gene but does not increase gene transcription can reduce the effect of a normal transcription factor by occupying promoter binding sites without increasing transcription.

It will be apparent to one skilled in the art that modification to the amino acid sequence of peptides agents could enhance the binding and/or stability of the peptide with respect to its target sequence. In addition, modification of the peptide may also increase the in vivo stability of the peptide thereby reducing the effective amount of peptide necessary to inhibit the activity of the polypeptides herein disclosed. This would advantageously reduce undesirable side effects which may result in vivo. Modifications include, by example and not by way of limitation, acetylation and amidation. Alternatively or preferably, said modification includes the use of modified amino acids in the production of recombinant or synthetic forms of peptides. It will be apparent to one skilled in the art that modified amino acids include, for example, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. Other modifications include amino acids with a C₂, C₃ or C₄ alkyl R group optionally substituted by 1, 2 or 3 substituents selected from halo (e.g. F, Br, I), hydroxy or C₁-C₄ alkoxy. It will also be apparent to one skilled in the art that peptides which retain p53 binding activity could be modified by cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363.

According to a further aspect of the invention there is provided a method to treat a subject for a cancer comprising administering an effective amount of an agent according to the invention.

In a preferred method of the invention said subject is human.

In a preferred method of the invention said cancer is prostate cancer.

According to a further aspect of the invention there is provided a method to immunise an animal against a cancerous condition comprising administering an effective amount of a nucleic acid or polypeptide encoded by a nucleic acid molecule selected from the group consisting of according to the invention.

In a preferred method of the invention said animal is a human.

In a further preferred embodiment of the invention said cancer is prostate cancer.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following Figures and Materials and Methods;

FIG. 1: Verification of CD133 as a stem cell marker of prostatic epithelia: 1A: A paraffin section of prostatic acini labelled with the nuclear stain DAPI (Blue) and anti-CD133 directly conjugated to PE (Red). 1B: Basal cells with the phenotype α₂β₁^{hi}/CD133⁺ have a higher colony forming efficiency (CFE) than α₂⊕₂^{low}/CD133⁻. (CFE) was calculated as the number of colonies formed per number of selected cells ×100%. CFEs are expressed as the ratio of the control CFE. Results show means ±s.e.m of four experiments. 1C. Xenografts of prostate acini formed by transplantation of α₂β₁^{hi}/CD133⁺ basal cells stained with (A) Haematoxylin and Eosin, (B) 34βE12, (C) anti-K18, (D) anti-PAP (E) Anti-androgen receptor. Bar 40 μm;

FIG. 2: Characterisation of tumour ‘stem’ cells from a lymph node metastasis of the prostate (LNMP). 2A. Tumour cells selected on the basis of □₂□₁/CD133 differentiate in culture. 2B. Invasion assay activity of LNMP in comparison to PC3M and an immortalised prostate epithelial cell line, PNT1a;

FIG. 3a represents NFkB Expression from α₂^{hi}/CD133+ cells (PE434 cells; Gleason 9); FIG. 3b represents a FACS dot plot of NFkB and CD133+ expression; 84% of cells are positive for NFkB alone and 0.21% are positive for NFkB and CD133.

Table 1 summarises array analysis of prostate stem cells and prostates cancer stem cells. Genes/nucleic acid and amino acid sequences are identified by Genbank accession number which can be accessed at http://www.ncbi.nlm.nih.gov. Genes are also identified by common name. The content of each accession entry is incorporated by reference and including the amino acid sequences of individual genes.

### Materials and Methods

### Genotype of Isolated Tumour Stem Cells

Using a combination of microsatellite markers associated with sporadic prostate cancer (8p 10q 16p) we can determine whether the isolated HEA⁺/CD44⁺/α₂β₁^{hi}/CD133⁺ cells display loss of heterozygosity patterns characteristic of prostate tumours in comparison to blood lymphocyte DNA from the same patient. The analysis is carried out on a microsampling of cultures with 3 MM paper and fluorescently labelled PCR primers (MacIntosh et al., 1998). This will enable us to discriminate between normal and cancer cells and determine whether stem cells are indeed targets for transforming events.

### Proliferative, Differentiative and Malignant Potential of Putative Cancer Stem Cells

Distinct populations of tumour cells are isolated and their proliferative, differentiative and malignant potential determined in vitro and in vivo. The following populations (HEA⁺/CD44⁻ (luminal cells), HEA+/CD44⁺ (basal cells), HEA⁺/CD44⁺/α₂β₁^{low}/CD133⁻ (transit cells), HEA⁺/CD44⁺/α₂β₁^{hi}/CD133⁺ (stem cells) are isolated and compared with the unsorted tumour population.

Colony Forming efficiency (CFE): Anchorage Independent and Anchorage-Dependent Growth

The transforming potential of distinct populations (as above) of cancer cells (anchorage independence) is measured by their ability to form colonies in soft agar. Individual colonies are counted after 21 days using an inverted microscope. Comparisons are made of CFE and colony size.

### Morphogenesis in Gels of Reconstituted Basement Membrane Matrix

We have determined the potential of tumour stem cells and their progenitors to undergo glandular morphogenesis in reconstituted basement membrane (e.g. Matrigel). We have demonstrated that normal basal cells can undergo glandular morphogenesis when grown in a collagen based matrix, (e.g. Matrigel) with stroma, in the presence of androgens. Spheroids are generated which are architecturally and phenotypically similar to in vivo acini and are often branched alveolar- and duct like (Lang et al., 2001). In contrast, cancer cells often form large aggregates of spindle-shaped cells with no obvious organisation. Nonetheless, the structures will often contain cells that show some degree of differentiation and can be compared to the original tumour.

### Invasion Assays

The ability of these stem cells to migrate across Matrigel is determined by the modified Boyden-chamber method (Albini et al., 1987). Migration rates will be evaluated using time-lapse confocal microscopy, using cells labelled with EGFP. We have generated prostate epithelium expressing low levels of EGFP. Recombinant retrovirus based on pLNCX-EGFP(2) generated will be used to infect the cell populations and G418 resistant colonies will be used in motility assays. The low levels of GFP expression will be used to track invasion and motility in real time.

### In Vivo Tumourigenesis

Tumour stem cells must possess key criteria that define normal stem cells: after transplantation they must proliferate, differentiate and self-renew. To determine the ability of distinct tumour phenotypes, to colonise in vivo, grafts of stem cells, transit cells, basal cells, luminal cells and unsorted cells are introduced into the prostates of 6 to 8 week old male, immuno-compromised mice. The mice are treated hormonally at the time of grafting by subcutaneous implantation of sustained release testosterone pellets. The number of cells from each population that successfully engraft and initiate tumour proliferation is determined by varying the number of cells implanted. The self-renewal capacity of the distinct populations is determined by transplanting serially into secondary recipients.

### Array Sample and Data Processing

### Total RNA Extraction

α₂^{high}/CD133⁺ Cells

Total RNA is extracted from up to 1×10⁴ selected cells using QIAgen RNeasy micro columns. Cells are lysed in 100 μl RLT buffer+1% β-mercaptoethanol and the manufactures protocol for “total RNA isolation from animal cells” is followed (RNeasy_Micro0403.pdf, pages 39-44, which is incorporated by reference).

### α₂^{low} Low Cells

Total RNA is extracted from between 1×10⁵ and 1×10⁶ selected cells using QIAgen RNeasy mini columns. Cells are lysed in 350 μl RLT buffer+1% β-mercaptoethanol and the manufactures protocol for “isolation of total RNA from animal cells” cells is followed (RNeasy_Mini0601.pdf, pages 31-35, which is incorporated by reference).

RNA yields are determined spectrophotometrically at 260 nm and RNA integrity checked by capillary electrophoresis using an Agilent 2100 bioanalyzer.

Production of Fragmented Labelled cRNA

Total RNA is amplified using two rounds of cDNA synthesis and IVT (in vitro transcription) and biotin labelled by following the Affymetrix small scale labelling protocol vII (smallv2_technote.pdf which is incorporated by reference) with the following modifications:
1. 10-50 ng of total RNA is used per sample (step 1).
2. T4 DNA polymerase steps in the two second strand cDNA synthesis reactions are omitted (steps 2 & 7).
3. Second cycle, IVT for cRNA amplification and labelling (step 9) uses the Affymetrix GeneChip IVT labelling ldt instead of the ENZO BioArray HighYield RNA transcript labelling kit and the Affymetrix eukaryotic sample and array processing standard protocol (expression_s2_manual_{—}0604.pdf, section 2.1.34-2.1.35 which is incorporated by reference) is followed for this stage.

The quality of first and second round cRNA products and fragmented cRNA are checked by capillary electrophoresis using an Agilent 2100 bioanalyzer.

### Array Hybridisation

Labelled fragmented cRNA (15 μg) is hybridised to oligonucleotide probes on an Affymetrix HG-U133plus2 GeneChip. For hybridisation, washing, staining and scanning the Affymetrix eukaryotic sample and array processing standard protocol (expression_s2_manual_{—}0604.pdf, section 2.2.3-2.3.17 which is incorporated by reference) is followed.
1. Hybridisation is conducted using an Affymetrix Hybridisation Oven 640.
2. Washing and staining stages are conducted using an Affymetrix Fluidics Station 450 using the EukGE-WS2v5 protocol.
3. Scanning of arrays is done with an Affymetrix Gene Scanner 3000.

### Data Processing

Scanned GeneChip images are processed using Affymetrix GCOS software to derive an intensity value and flag (present, absent or marginal) for each probe. Probe intensities are derived using the MASS algorithm.

Comparisons between different sample datasets are conducted using Agilent GeneSpring GX software. Datasets to be compared are first normalised using three steps (consecutively applied in the order given):
1. Transform values <0.01 to 0.01
2. Normalise each chip to the 50 percentile of the measurements taken for that chip.
3. Normalise each probe to the median of the measurements for that probe.

For the purpose of analysing the data the following parameters are applied:
1. All cells derived from prostate cancer specimens are classed malignant, all cells derived from BPH specimens are classed benign (“tumour type” parameter)
2. All cells obtained by selection for high integrin α₂β₁ expression and CD133 are classed stem cells, all cells selected for low integrin α₂β₁ expression are classed committed basal cells (“cell type” parameter).
3. Further interpretations of the data allow for the combining of the above parameters to derive the conditions: malignant stem cells, malignant committed basal cells, benign stem cells and benign committed basal cells.

Low quality and uninformative data is removed using three selections (consecutively applied in the order given):
1. Remove probes flagged “absent” in all samples.
2. Remove probes with standard deviation within a parameter class of >1 in at least 3 of the 4 conditions.
3. Remove probes with less than a 2-fold overall change in normalised expression value between all four of the conditions.

1. An agent that modulates the activity of a cancer stem cell specific nucleic acid molecule, or a polypeptide encoded by a cancer stem nucleic acid molecule, wherein said cancer cell specific nucleic acid molecule is selected from the group consisting of:
i) a nucleic acid molecule consisting of a nucleic acid sequence as represented in SEQ ID NO: 1-452;
ii) a nucleic acid molecule that hybridises under stringent hybridisation conditions to the nucleic acid molecule in (i) above and which encodes a polypeptide wherein said polypeptide is stem cell specific for use as a pharmaceutical.

2. An agent according to claim 1 wherein said agent is an antagonist.

3. An agent according to claim 1 wherein said agent is an agonist.

4. An agent that modulates the activity of a polypeptide comprising an amino acid sequence encoded by a nucleic acid molecule selected from the group consisting of:
i) a polypeptide encoded by a nucleic acid molecule as represented by the nucleic acid sequence in SEQ ID NO: 1-452;
ii) a polypeptide encoded by a a nucleic acid molecule that encodes a variant polypeptide wherein said variant polypeptide is modified by addition, deletion or substitution of at least one amino acid residue of the amino acid sequence encoded by a nucleic acid sequence selected from the group consisting of SEQ ID NO: 1-452 wherein said polypeptide is stem cell specific characterized in that said agent is for use as a pharmaceutical.

5. An agent according to claim 4 wherein said agent is a polypeptide.

6. An agent according to claim 5 wherein said polypeptide is an antibody or an active binding part of an antibody.

7. An agent according to claim 6 wherein said antibody is a monoclonal antibody or active binding part thereof.

8. An agent according to claim 6 wherein said antibody is a chimeric antibody or a humanised antibody.

9. An agent according to claim 6, wherein said agent is an antibody fragment.

10. An agent according to claim 9 wherein said fragment is selected from the group consisting of: Fab, Fab₂, F(ab′)₂, Fv, Fc, Fd, or scFvs.

11. An agent according to claim 10 wherein said antibody fragment is a single chain antibody variable region fragment.

12. An agent according to claim 10 wherein said antibody is bispecific binding two epitopes of two different antigens.

13. An agent according to claim 6 wherein said antibody is an opsonic antibody.

14. An agent according to claim 6, wherein said antibody is provided with a marker or tag.

15. An agent according to claim 6 wherein said antibody, or antibody fragment, has associated therewith or crosslinked thereto a therapeutic agent.

16. An agent according to claim 15 wherein said therapeutic agent is a chemotherapeutic agent.

17. An agent according to claim 15 wherein said agent is selected from the group consisting of: cisplatin; carboplatin; cyclophosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; and camptothecin.

18. An agent according to claim 1, wherein said agent is a nucleic acid molecule.

19. An agent according to claim 18 wherein said nucleic acid molecule is selected from the group consisting of: an antisense nucleic acid, an aptamer, or a small interfering RNA.

20. An agent according to claim 19 wherein said nucleic acid molecule is a small interfering RNA.

21. An agent according to claim 20 wherein said small interfering RNA is 21-29 nucleotides in length.

22. A pharmaceutical composition comprising an agent according to claim 1.

23. A composition comprising a nucleic acid molecule selected from the group consisting of:
i) a nucleic acid molecule consisting of a nucleic acid sequence as represented in SEQ ID NO: 1-452;
ii) a nucleic acid molecule that hybridizes under stringent hybridization conditions to the nucleic acid molecule in (i) above and which encodes a polypeptide wherein said polypeptide is stem cell specific for use as a vaccine.

24. A composition comprising a polypeptide comprising an amino acid sequence encoded by a nucleic acid molecule as represented in SEQ ID NO 1-452, or a variant polypeptide wherein said variant is modified by addition, deletion or substitution of at least one amino acid residue of the amino acid sequence encoded by a nucleic acid molecule as represented in SEQ ID NO 1-452 for use as a vaccine.

25. A composition according to claim 24 wherein said composition includes an adjuvant and/or a carrier.

26. A vector which is adapted for the expression of a humanised or chimeric antibody or an antibody fragment according to claim 8.

27. A cell which has been transformed or transfected with the vector according to claim 26 encoding the humanised or chimeric antibody or an antibody fragment.

28. A method for the production of the humanised or chimeric antibody or an antibody fragment comprising:
i) providing a cell transformed or transfected with a vector which comprises a nucleic acid molecule encoding the humanised or chimeric antibody according to claim 8;
ii) growing said cell in conditions conducive to the manufacture of said antibody; and
iii) purifying said antibody from said cell, or its growth environment.

29. A hybridoma cell line which produces a monoclonal antibody according to claim 7.

30. A method of producing monoclonal antibodies according to claim 7 comprising using hybridoma cell line which produces a monoclonal antibody.

31. A method for preparing a hybridoma cell-line producing a monoclonal antibody comprising the steps of:
i) immunizing an immunocompetent mammal with an immunogen comprising at least one polypeptide having the amino acid sequence encoded by a nucleic acid molecule comprising a nucleic acid sequence as represented in SEQ ID NO 1-452, or fragments thereof;
ii) fusing lymphocytes of the immunized immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

32. A diagnostic assay for the determination of cancer in a subject comprising the steps of:
i) providing an isolated cell sample;
ii) contacting the sample in (i) with a binding agent(s) that binds to a nucleic acid molecule as represented by the nucleic acid sequence in SEQ ID NO 1-452, or a nucleic acid molecule that hybridizes to said nucleic acid molecule under stringent hybridization conditions and encodes a variant polypeptide; and
iii) determining the expression of said nucleic acid molecule in said sample when compared to a normal matched control sample.

33. An assay according to claim 32 wherein said binding agent(s) is an oligonucleotide primer.

34. An assay according to claim 33 wherein said assay is a polymerase chain reaction.

35. An assay according to claim 32 wherein said binding agent is an antibody that specifically binds a polypeptide encoded by a nucleic acid molecule as represented by the nucleic acid sequence in SEQ ID NO 1-452, or a polypeptide variant comprising an amino acid sequence that varies from a reference amino acid sequence by addition, deletion or substitution of at least one amino acid residue.

36. An assay according to claim 35 wherein said cancer is prostate cancer.

37. A kit comprising a binding agent specifically reactive with a nucleic acid molecule as represented by the nucleic acid sequence in SEQ ID NO 1-452, or an agent specifically reactive with a polypeptide comprising an amino acid sequence encoded by a nucleic acid molecule comprising a nucleic acid sequence as represented in SEQ ID NO 1-452.

38. A kit according to claim 37 wherein said kit further comprises an oligonucleotide or antibody specifically reactive with said nucleic acid molecule or said polypeptide.

39. A kit according to claim 37 wherein said kit comprises a thermostable DNA polymerase and components required for conducting the amplification of nucleic acid.

40. A kit according to claim 37, wherein said kit includes a set of instructions for conducting said polymerase chain reaction and control nucleic acid.

41. A kit according to claim 37, wherein said kit comprises an antibody specifically reactive with said polypeptide.

42. A kit according to claim 41 wherein said kit comprises components required for conducting an immunoassay including, for example, a secondary antibody specifically reactive with a primary antibody that specifically binds said polypeptide(s) and enzyme reagents required to detect the binding of said secondary antibody with said primary antibody.

43. A method to screen for an agent that modulates the activity of a polypeptide encoded by a nucleic acid molecule selected from the group consisting of:
a) a nucleic acid molecule consisting of a nucleic acid sequence as represented in SEQ ID NO 1-452;
b) a nucleic acid molecule that hybridizes under stringent hybridization conditions to the nucleic acid molecule in (i) above and which encodes a polypeptide wherein said polypeptide is stem cell specific;
said method comprising:
i) forming a preparation comprising a polypeptide, or sequence variant thereof, and at least one agent to be tested;
ii) determining the activity of said agent with respect to the activity of said polypeptide.

44. A method according to claim 43 wherein said agent is an antagonist.

45. A method according to claim 43 wherein said agent is an agonist.

46. A method to treat a subject for a cancer comprising administering an effective amount of an agent according to claim 1.

47. A method according to claim 46 wherein said subject is human.

48. A method according to claim 47 wherein said cancer is prostate cancer.

49. A method to immunise an animal against a cancerous condition comprising administering an effective amount of a nucleic acid or polypeptide encoded by a nucleic acid molecule selected from the group consisting of:
i) a nucleic acid molecule consisting of a nucleic acid sequence as represented in SEQ ID NO 1-452;
ii) a nucleic acid molecule that hybridizes under stringent hybridization conditions to the nucleic acid molecule in (i) above and which encodes a polypeptide wherein said polypeptide is stem cell specific.

50. A method according to claim 49 wherein said animal is a human.

51. A method according to claim 50 wherein said cancer is prostate cancer.
